(19) United States
(12) Patent Application Publication
(10) Pub. No.: US 2010/0233209 A1
(43) Pub. Date: Sep. 16, 2010

(54) Chikungunya virus infectious clones and uses therefor

(76) Inventors: Stephen T. Higgs, Dickinson, TX (US); Dana L. Vanlandingham, Dickinson, TX (US); Konstantin Tsetsarkin, Glaveston, TX (US)

Correspondence Address: Adler & Associates, 8011 Candle Lane, Houston, TX, 77071, US

(21) Appl. No.: 11/990,362

(22) PCT Filed: Aug. 11, 2006
(86) PCT No.: PCT/US06/31432

§ 371 Date: Feb. 11, 2008

Related U.S. Application Data
(60) Provisional application No. 60/707,442, filed on Aug. 11, 2005.

(51) Int. Cl. A61K 39/12; C12N 15/63; C12N 5/10; C07H 21/04; C12Q 1/68; C12Q 1/70; A61P 31/12

(52) U.S. Cl. 424/218.1; 435/320.1; 435/325; 536/23.72; 435/6; 435/5

(57) ABSTRACT

The present invention developed and characterized in vitro and in vivo three full-length cDNA clones based on the alphavirus chikungunya, two sets of infectious clones based on CHIKV and replicons based on the principle used to generate the infection clones. Described herein is the method to generate such infective clones and replicons, their composition and their use as molecular tool, a delivery vehicle and vaccine.

### CROSS REFERENCE TO RELATED APPLICATION

This U.S. National Stage application claims benefit of priority under 35 U.S.C. 365 of international application PCT/US2006/031432, filed Aug. 11, 2006, now abandoned, which claims benefit of priority under 35 U.S.C. 119(e) of provisional U.S. Ser. No. 60/707,442, filed Aug. 11, 2005, now abandoned.

### FEDERAL FUNDING LEGEND

This invention was produced using funds obtained through grant RO1-AI47877 from the National Institutes of Health. Consequently, the federal government has certain rights in this invention.

### BACKGROUND OF THE INVENTION

1. Field of the Invention

The present invention relates to the fields of molecular biology, virology and immunology. More specifically, the present invention provides a viral expression system comprising the nucleotide sequence of alphavirus chikungunya (strain 37997 and other isolates including those from LaReunion) (CHIKV) and discloses its use as a molecular tool, a delivery vehicle and vaccine.

2. Description of the Related Art

CHIKV viruses are arthropod-borne viruses in the family Togaviridae. These viruses are known to be responsible for outbreaks especially during 2005-2006 in the Indian Ocean. These viruses consist of a positive sense, linear, ssRNA genome which is ˜11.7 Kb in size. The 5′ terminus of the virus is capped and the 3′ terminus is polyadenylated. The nonstructural proteins (nsP1-4) are encoded at the 5′ end of the genome followed by the structural proteins which are encoded from a subgenomic promoter at the 3′ end. The structural proteins consist of a capsid, two envelope glycoproteins (E1 and E2), and two small peptides, E3 and 6K (Strauss and Strauss 1994).

The two genera in the family Togaviridae are rubivirus, whose only member is Rubella virus, and Alphavirus (Schlesinger and Schlesinger 2001). The 26 species of Alphaviruses have been grouped together based on high amino acid sequence identity in the nonstructural and structural proteins and based on antigenic relationships (Schlesinger and Schlesinger 2001; Hart 2001). Some viruses in the genus Alphavirus include: Venezuelan equine encephalitis virus (VEEV), eastern equine encephalitis virus (EEEV), western equine encephalitis virus (WEEV), Sindbis Virus (SINV), and Ross River virus (RRV), ONNV and CHIKV. SINV is the alphavirus that has been studied most extensively used as a model for alphavirus structure, replication and in the determination of the function of various genes. SINV is in the western equine encephalitis antigenic complex and CHIKV is from the Semliki Forest antigenic complex, however, these viruses belong to the Alphavirus genus and the genome functions and gene interactions are assumed to be similar (Table 1). The present invention focuses on CHIKV which is in the Semliki Forest antigenic complex.

TABLE 1
Functions of different alphavirus genes.

| | Nucleotide | Function and | |
|---|---|---|---|
| Gene/Region | ONNV | Additional Information | Gene Interactions |
| 5′NCR | 1-79 | Initiation of replication | 3′NCR |
| | | Minus and plus strand RNA | |
| | | synthesis | |
| | | Important determinant of virule [symbol] | |
| nsP1 | 80-1684 | Synthesis of minus strand RN [symbol] | modulates activity of |
| | | Caps genomic and subgenomi [symbol] | proteinase of nsP2 |
| | | RNA with Methyltransferase a [symbol] | interacts with nsP4 |
| | | Guanyltransferase | |
| nsP2 | 1685-4078 | RNA helicase during RNA | |
| | | replication | |
| | | and transcription | |
| | | Nonstructural proteinase | |
| | | Synthesis of 26S subgenomi [symbol] | |
| | | mRNA | |
| nsP3 | 4079-5767 | No sequence motif similar in other RNA virus genome [symbol] | |
| nsP4 | 5768-7618 | | |
| Capsid | 7668-8447 | Virion nucleocapsid | Interacts with PE2 and |
| | | Binds to the viral genomic RN [symbol] | |
| PE2 | | cleaved late in vertebrate cel [symbol] l | E1 to form stable |
| | | | heterodimers |
| E3 | 8448-8639 | Formed from cleavage of PE [symbol] | |
| E2 | 8640-9908 | Transmembrane glycoprotei [symbol] | Cytoplasmic domain |
| | | Important determinants of virule [symbol] | interaction with capsid [symbol] |
| | | Protein that interacts with cellu [symbol] | important for virus |
| | | receptors | assembly, leading to vir [symbol] |
| | | Formed from cleavage of PE [symbol] | budding from the cel [symbol] |
| | | | surface |
| 6K | 9909-10091 | needed for budding | |

| | | |
|---|---|---|
| E1 | 10092-11408 | PE2 and E2 heterodim [symbol] |

| | | |
|---|---|---|
| 3′NCR | Plus strand RNA synthesis | 5′UTR |

[symbol]

indicates data missing or illegible when filed

### Alphavirus Virion Structure

Alphaviruses consist of an icosahedral nucleocapsid coated with a lipid envelope. The two surface glycoproteins, E1 and E2, form heterodimers which are embedded in the envelope. The heterodimers are organized as trimers that make up the majority of the outer surface of the virion. The envelope consists of a lipid bilayer which is derived from the plasma membrane of the host cell. The capsid is found inside the envelope and surrounds the viral RNA genome.

Thus, the structure of this virus enables the construction of either full length infectious clones, full length infectious clones that also contain a 26S subgenomic promoter that can be used to express heterologous genes, or to divide the plasmid into two or three plasmids which causes the virus to be infectious but unable to replicate.

A full length cDNA clone of the alphavirus SINV (dsSIN), pTE/3′2J, was developed as a transient expression system for heterologous RNAs and proteins and has proved to be an efficient expression system in cell culture and mosquitoes (Hahn et al. 1992; Higgs et al. 1995). A number of gene sequences have since been delivered to mosquitoes using this and other dsSIN expression systems including: antisense constructs to interfere with viral replication (Powers et al. 1996; Olson et al. 1996; Higgs et al. 1998; Adelman et al. 2001), to silence genes such as luciferase (Johnson et al. 1999), and mosquito genes such as prophenoloxidase (Shiao et al. 2001; Tamang et al. 2004); genes to over-express toxin genes (Higgs et al. 1995), single chain antibodies, (James et al. 1999), and to knockout of endogenous genes using RNAi (Attardo et al. 2003). The dsSIN pTE/3′2J system has also been used to infect larval arthropods by feeding infected cells that expressed green fluorescent protein (GFP) or defensin genes (Higgs et al. 1999; Cheng et al. 2001). In addition to the GFP reporter system, other reporters have been used such as chloramphenicol acetyltransferase (Olson et al. 1994).

Despite the successful employment of the dsSIN expression system, a short coming of the original system was the relatively low efficiency with which it infects and disseminates from midgut following oral infection. Although this has been addressed using various strategies, the oral infectivity and dissemination rates of dsSIN expression systems are frequently too low for use with genes which are difficult to characterize in Ae. aegypti.

Thus, prior art is deficient in a chikungunya virus-based viral expression system that can express immunogenic nucleotide sequences in vertebrates and can express nucleotides of interest in invertebrates and vertebrates. The present invention fulfills this long-standing need and desire in the art.

### SUMMARY OF THE INVENTION

A full length CHIKV infectious clone was found to express genes inserted either 5′ or 3′ of the structural genes at a higher rate than previous systems such as Sindbis virus (SINV). This infectious clone expressed genes at a high rate in vitro using vertebrate and invertebrate cells but also in vivo. It was also highly immunogenic in vertebrates and typically only caused morbidity, not mortality. This was in contrast with the Venezuelan equine encephalitis based expression system.

The present invention is directed to the development and characterization of two groups of novel expression systems based on Chikungunya virus 37997 and other isolates including those from LaReunion which are infectious to vertebrates and invertebrates. The first group of expression systems contains the full length CHIKV genome. Additionally, by adding another promoter to the clone that expresses full-length sequence of CHIKV, one can insert sequences, for example of visible marker genes that would enable tracking the virus as it replicates and disseminates in the mosquito. Thus, one clone is the exact sequence of CHIKV (strain 37997) and other isolates including those from LaReunion, another clone expresses a gene of interest using a subgenomic promoter (26S) located at the 5′ position to the structural genes and the third plasmid can express a gene of interest at the 3′ position to the structural genes.

The second group of infectious clones, based on the CHIKV (37997) and other isolates including those from LaReunion, is infectious to vertebrates but is deficient in the ability to undergo replication. The removal of the structural genes into either one or two separate plasmids, referred to as the helper plasmids, with the nonstructural genes on another plasmid allows this construct to be used as a vehicle to deliver immunogenic nucleotides to vertebrates. The helper either contains all the structural genes with a second subgenomic promoter to express an inserted immunogenic gene sequence of interest or a plasmid containing the capsid genes of CHIKV with the remaining structural genes on a third plasmid. The helper plasmid contains the sequence for the 26S subgenomic promoter upstream of a multiple cloning site to enable expression of immunogenic heterogeneous RNA.

It is contemplated that these constructs will be initially more infectious and produce a highly immunogenic response when used as a vehicle to deliver immunogenic RNAS in vertebrates as compared to previous systems. Additionally, expression of heterogeneous RNAs in invertebrates using the full length CHIKV infectious clones will be a dramatic improvement over previous expression system. This system has been found to produce higher levels of infection, dissemination in mosquitoes and the expression of EGFP from an epidemiologically important virus in Ae. aegypti and Ae. albopictus, this system is a significant improvement over the SINV system for the study of virus-vector relationships with Ae. aegypti and Ae. albopictus mosquitoes. These full length CHIKV infectious clones are orally infectious in Ae. aegypti and Ae. albopictus with high infection and dissemination rates.

It is contemplated that the ability of the 5′CHIK EGFP virus to express a heterologous gene in 100% of mosquito's midguts and to disseminate to 90% of the salivary glands and head tissues, following oral infection will enable the biological characterization of endogenous genes. Naturally occurring CHIKV causes large epidemics and with apparently numerous human cases of laboratory infections. This virus is different from other Alphaviruses that are thought to be useful as vaccine vehicles because CHIKV is infectious and causes an immune response but does not normally cause death. It is contemplated that these clones will be more acceptable for use as a vaccine because they do not typically cause mortality and yet are highly immunogenic in humans.

Thus, the clones produced in the present invention can be used to express nucleotides of interest, heterologous genes, genes for overexpression, genes for knockout/knockdown in both invertebrates and vertebrates to evaluate gene function in a variety of organisms. These clones can be used as a delivery vehicle for sequences with immunogenic properties that could stimulate the vertebrate immune system and induce protective immune response. Furthermore, genetic manipulation of these clones would attenuate them to produce virus that is infectious but has reduced virulence in vertebrates and invertebrates, thereby providing a vaccine vehicle for both CHIKV and for other etiologic agents.

In one embodiment of the present invention, there is provided an expression vector that comprises a DNA sequence encoding a full-length chikungunya virus (CHIKV) comprising nonstructural protein genes and structural protein genes of the CHIKV. In a further related embodiment of the present invention, there is provided a host cell comprising and expressing the vector that comprises a DNA sequence encoding the expression vector described herein.

Additionally, in further embodiments of the present invention, there is an infectious clone comprising the DNA encoding a chikungunya virus (CHIKV) described supra, a pharmaceutical composition comprising the attenuated chikungunya virus encoded by the infectious clone, a DNA sequence encoding a protein of interest expressed by the clone and a pharmaceutically acceptable carrier, an immunogenic composition comprising a live attenuated chikungunya virus encoded by the infectious clone and a DNA sequence encoding an immunogenic peptide expressed by the clone and an immunogenic composition comprising an attenuated chikungunya virus encoded by the infectious clone, where the attenuated CHIKV is inactivated and a DNA sequence encoding an immunogenic peptide expressed by the clone.

In yet another embodiment of the present invention, there is provided a method of evaluating the function of a gene in an organism. This method comprises expressing the gene or knocking out the gene of interest using the clone described herein and determining the effect of over-expression or knocking out the gene in the organism. Thus, evaluating the function of the gene in the organism.

In yet another embodiment of the present invention, there is provided a method of inducing protective immune response in a subject. This method comprises administering pharmacologically effective amounts of an immunogenic composition comprising chikungunya virus encoded by the clone described supra and a DNA sequence encoding an immunogenic peptide expressed by the clone. Thus, a protective immune response is induced in the subject.

In yet another embodiment of the present invention, there is provided a method of inducing a protective immune response in a subject. This method comprises administering pharmacologically effective amounts of an immunogenic composition comprising chikungunya virus encoded by the clone described herein, where the attenuated CHIKV is inactivated and a DNA sequence encoding an immunogenic peptide expressed by the clone. Thus, inducing protective immune response in the subject.

In another embodiment of the present invention, there is provided an expression vector that comprises a DNA sequence encoding a full-length chikungunya virus (CHIKV) comprising non-structural protein genes and structural protein genes of the CHIKV and an additional subgenomic promoter.

In a further related embodiment of the present invention, there is provided a host cell comprising and expressing an expression vector that comprises a DNA sequence encoding a full-length chikungunya virus (CHIKV) and an additional subgenomic promoter.

Additionally, in further embodiments of the present invention, there is provided an infectious clone comprising the DNA encoding a chikungunya virus and the additional sub-genomic promoter described supra, a pharmaceutical composition comprising the attenuated chikungunya virus and the sub-genomic promoter encoded by the infectious clone described supra, a DNA sequence encoding a protein of interest expressed by the clone and a pharmaceutically acceptable carrier, an immunogenic composition comprising a live attenuated chikungunya virus and the sub-genomic promoter encoded by the infectious clone and a DNA sequence encoding an immunogenic peptide expressed by the clone and an immunogenic composition comprising an attenuated chikungunya virus and the sub-genomic promoter encoded by the infectious clone, where the attenuated CHIKV is inactivated and a DNA sequence encoding an immunogenic peptide expressed by the clone.

In another embodiment of the present invention, there is provided a method of evaluating function of a gene in an organism. Such a method comprises expressing the gene or knocking out the gene of interest using the infectious clone that comprises a DNA sequence encoding a full-length chikungunya virus and an additional subgenomic promoter. This is followed by determining the effect of over-expressing or knocking out the gene in the organism, thereby evaluating the function of the gene in the organism.

In yet another embodiment of the present invention, there is a method of inducing protective immune response in a subject. This method comprises administering pharmacologically effective amounts of an immunogenic composition comprising attenuated chikungunya virus and a sub-genomic promoter encoded by the clone described supra and a DNA sequence encoding an immunogenic peptide expressed by the clone. Thus, inducing protective immune response in the subject.

In yet another embodiment of the present invention, there is a method of inducing a protective immune response in a subject. This method comprises administering pharmacologically effective amounts of an immunogenic composition comprising an attenuated chikungunya virus and a sub-genomic promoter encoded by the clone described herein, where the attenuated CHIKV is inactivated and a DNA sequence encoding an immunogenic peptide expressed by the clone. Thus, inducing protective immune response in the subject.

In another embodiment of the present invention, there is a CHIKV replicon system. This system comprises a replicon comprising non-structural genes of the CHIKV and a marker gene. Additionally, this system also comprises a helper system comprising structural genes of the CHIKV. In another related embodiment of the present invention, there is a host cell comprising and expressing the replicon system discussed herein.

In another related embodiment of the present invention, there is provided a virus like particle. This virus like particle comprises genes encoded by the replicon system discussed supra.

In yet another related embodiment of the present invention, there is provided a method of identifying sites of primary CHIKV infection in a mosquito vector. This method comprises feeding the virus like protein discussed supra to the mosquito vector and detecting expression of the marker gene in the midgut and salivary gland of the mosquito vector, thereby identifying sites of primary CHIKV infection in the mosquito vector.

### BRIEF DESCRIPTION OF THE DRAWINGS

FIG. 1 shows the construction of 5′ pCHIKic EGFP.

FIG. 2 compares the in vitro growth of CHIKV strain 37997 and virus produced from pCHIKic in Vero, C6/36 and MOS-55 cell lines.

FIGS. 3A-3J show EGFP expression on days 7 and 14 p.i. with virus derived from 5′ and 3′ pCHIKic EGFP in midguts, salivary glands and eyes. FIG. 3A shows 3′CHIKV EGFP on day 7 p.i. midgut, FIG. 3B shows 3′CHIKV EGFP on day 7 p.i. salivary gland, FIG. 3C shows 3′CHIKV EGFP on day 14 p.i. midgut, FIG. 3D shows 3′CHIKV EGFP on day 14 p.i. salivary gland, FIG. 3E shows 3′CHIKV EGFP on day 14 p.i. eyes, FIG. 3F shows 5′CHIKV EGFP on day 7 p.i. midgut, FIG. 3G shows 5′CHIKV EGFP on day 7 p.i. salivary gland, FIG. 3H shows 5′CHIKV EGFP on day 14 p.i. midgut, FIG. 3I shows 5′CHIKV EGFP on day 14 p.i. salivary gland and FIG. 3J shows 5′CHIKV EGFP on day 14 p.i. eyes.

FIGS. 4A-4G show the map and the sequence of the plasmid: pChik-3 that contains 14608 base pairs (SEQ ID NO: 1).

FIGS. 5A-5H show the map and the sequence of the plasmid: p5′Chik-37997ic that contains 15470 base pairs (SEQ ID NO: 2).

FIGS. 6A-6H show the map and the sequence of the plasmid pChik-3′ GFP that contains 15769 base pairs (SEQ ID NO: 3).

FIGS. 7A-7B show the construction of CHIKV replicon system and the dynamic of accumulation of infectious units. FIG. 7A is a diagrammatic representation of construction of CHIKV replicon and helper system. FIG. 7B shows the titer of packaged CHIKV replicons after RNA transfection into BHK-21 cells.

FIG. 8 compares fluorescence in BHK-21 cells that were transfected with either replicon RNA alone or with replicon and helper RNA and allowed to attach to 70% confluent monolayer of BHK-21 cells.

FIG. 9 shows infection of Vero (top), C6/36 (middle) and Mos55 (bottom) at an MOI of 1 for CHIK37997 VLP.

FIGS. 10A-10B show structure of CHIKV-LR and GFP expression in BHK-21 cells. FIG. 10A is a schematic representation of CHIKV-LR ic. FIG. 10B shows the genome structure of the CHIKV-LR 5′GFP and CHIKV-LR 3′GFP viruses. FIG. 10C shows GFP expression in BHK-21 cells under fluorescent microscope 24 h after electroporation of RNAs produced from CHIKV-LR 5′GFP or CHIKV-LR 3′GFP.

FIGS. 11A-11B show the in vitro growth curves of CHIKV strain LR2006 OPY1 (CHIKV-LR) and viruses produced from CHIKV-LR ic, CHIKV-LR5′GFP and CHIKV-LR 3′GFP in different cell cultures. FIG. 11A shows the invitro growth curves of these viruses in Vero cells and FIG. 11B shows the invitro growth curves of these viruses in C6/36 cells.

FIGS. 12A-12B show titers of ds viruses following serial passage in different cell lines. FIG. 12A shows titers of these viruses in BHK-21 cells and FIG. 12B shows titers of these viruses in C6/36 cells. In these figures: F, titer of GFP positive foci/mL; CPE, titer of plaque forming units/mL.

FIGS. 13A-13E show GFP expression in mosquitoes post-infection. GFP expression in Ae. aegypti (FIGS. 13A-13B) and Ae. albopictus (FIGS. 13C, 13D, 13E) mosquitoes when infected with viruses derived from CHIKV-LR 5′GFP (FIGS. 13A-13D) or CHIKV-LR 3′GFP (FIG. 13E) on day 14 p.i. FIGS. 13A, 13C and 13E show expression in mosquito midguts; FIGS. 13D and 13B show expression in mosquito salivary glands.

FIGS. 14A-14D show the effect of E1-A226V mutation on CHIKV-GFP viruses Ae. albopictus and Ae. aegypti infectivity. Percent of orally infected Ae. albopictus (FIGS. 14A, 14B) and Ae. aegypti (FIGS. 14C, 14D) mosquitoes presented with blood meals containing various concentrations of eGFP-expressing CHIK viruses. Serial 10-fold dilutions of viruses in the backbone of Reunion (LR-GFP-226V and LR-GFP-226A) (FIGS. 14A, 14C) and 37997 (37997-GFP-226A and 37997-GFP-226V) (FIGS. 14B, 14D) strains of CHIKV were made in L-15 medium followed by mixing the samples with defibrinated sheep blood. Mosquitoes were dissected at 7 dpi and eGFP expression in infected midguts was analyzed by fluorescence microscopy. A mosquito was considered infected if at least one foci of eGFP-expressing cells was present in the midgut. The experiments were performed twice for each virus (I and II).

FIGS. 15A-15C show the results of virus competition experiments. FIG. 15A is a schematic representation of competition experiments. FIG. 15B shows competition between LR-ApaI-226V and LR-226A viruses for colonization of midgut cells in Ae. albopictus mosquito and FIG. 15C shows competition between LR-ApaI-226V and LR-226A viruses for colonization of midgut cells in Ae. aegypti mosquito. BM, initial ratio of LR-ApaI-226V and LR-226A in blood meal samples. 1-4 ratio of LR-ApaI-226V and LR-226A RNA in four independent replicas of the eight to ten midguts per replica. Relative fitness (RF₁) of LR-Apa-226V to LR-226A was calculated as a ratio between 226V and 226A bands in the sample, divided by the control ratio between 226V and 226A in the blood meal. Relative fitness (RF₂) of LR-226A to LR-Apa-226V was calculated as a ratio between 226A and 226V bands in the sample, divided by the control ratio between 226A and 226V in the blood meal. Results are expressed as the average of four replicas±standard deviation (SD).

FIGS. 16A-16D show the effect of E1-A226V mutation on CHIKV dissemination into salivary glands and heads of Ae. albopictus and Ae. aegypti mosquitoes. Ae. albopictus (FIG. 16A) and Ae. aegypti (FIG. 16C) mosquitoes were orally infected with LR-GFP-226V and LR-GFP-226A. At the indicated time points, 16-21 mosquitoes were dissected and salivary glands were analyzed for eGFP expression. Percent of dissemination was estimated as ratio of the number of mosquitoes with eGFP-positive salivary glands to the number of mosquitoes with eGFP-positive midguts. For Ae. albopictus, infectious blood meal titers were 5.95 and 6.52 Log₁₀TCID₅₀/ml for LR-GFP-226V and LR-GFP-226A, respectively. For Ae. aegypti, the infectious blood meal titer was 6.95 Log₁₀TCID₅₀/ml for LR-GFP-226V and LR-GFP-226A viruses. Dissemination rates were compared statistically by Fisher's exact test using SPSS version 11.5. Asterisk indicates p<0.05.

FIGS. 16B and 16D show competition between LR-ApaI-226V and LR-226A for dissemination into heads of Ae. albopictus and Ae. aegypti mosquitoes. 10⁷ pfu of LR-ApaI-226V and LR-226A were mixed and orally presented to Ae. albopictus (FIG. 16B) and Ae. aegypti (FIG. 16D). Viral RNAs were extracted from four pools of five heads collected at 12 dpi. RT-PCR products were digested with ApaI, separated in 2% agarose gel and gels were stained using ethidium bromide. BM, initial ratio of LR-ApaI-226V and LR-226A in blood meal samples. 1-4 ratio of LR-ApaI-226V and LR-226A RNA in four independent replicas of the five pooled heads per replica.

FIGS. 17A-17B show effect of E1-226V mutation on CHIKV kinetics of viral growth in bodies of Ae. albopictus mosquitoes. FIG. 17A shows virus production in orally infected Ae. albopictus mosquitoes. Infected mosquitoes were sampled at 0, 1, 2, 3, 5, 7 and 14 dpi and titrated on Vero cells to estimate average titer±standard deviation of eight whole mosquitoes. Differences in viral titers were analyzed by pairwise t-tests. Asterisk indicates p<0.05. FIG. 17B shows kinetics of competition between LR-ApaI-226V and LR-226A in bodies of Ae. albopictus mosquitoes. 10⁷ pfu of LR-ApaI-226V and LR-226A were mixed and orally presented to Ae. albopictus. Infected mosquitoes were sampled at 1, 3, 5, 7 an d14 dpi. For each time point, viral RNA was extracted from two pools of ten mosquitoes. BM, initial ration of LR-ApaI-226V and LR-226A in blood meal samples. RF-relative fitness of LR-ApaI-226V to LR-226A was calculated as a ratio between 226V and 226A bands in the sample, divided by the control ratio between 226V and 226A in the blood meal. Results expressed as average of two replicas±standard deviation.

FIGS. 18A-18C show effect of E1-226V mutation on CHIKV transmission by Ae. albopictus and Ae. aegypti mosquitoes. In FIG. 18A, six 2- to 3-day-old suckling mice (Swiss Webster) were subcutaneously infected with a 20-μl mixture of ˜25 pfu LR-Apa-226V and ˜25 pfu of LR-226A viruses. In FIGS. 18B and 18C, Ae. aegypti and Ae. albopictus mosquitoes were presented with a blood meal containing 10⁷ pfu/ml of LR-Apa-226V and 10⁷ pfu/ml of LR-226A viruses. At 13 dpi, ten to 15 mosquitoes were placed in separate paper cartons and starved for 24 h. The next day, the mosquitoes in each carton were presented with a 2- to 3-day-old suckling mouse (Swiss Webster). Mice were returned to their cage and sacrificed on day 3 post-exposure. Blood from each individual mouse (˜50 μl) was collected and immediately mixed with 450 μl of TRIzol reagent for RNA extraction. BM and inoc.—initial ratio of LR-ApaI-226V and LR 226A in blood meal samples and inoculum for subcutaneous infection. 1-6 ratio of LR-ApaI-226V and LR-226A RNA in six individual mice.

FIGS. 19A-19B show effect of E1-A226V Mutation on In Vitro Growth of CHIKV in Standard (FIG. 19A) and Cholesterol-Depleted (FIG. 19B) C6/36 cells. Cholesterol-depleted C6/36 cells were produced by five passages in L-15 medium containing 10% FBS treated with 2% CAB-O-Sil for 12 h at room temperature as previously described (Weinstein, 1979). Confluent monolayers of standard (FIG. 19A) and cholesterol-depleted (FIG. 19B) C6/36 cells were infected with LR-ApaI-226V, LR-226A, 37997-226A and 37997-226V viruses at an MOI of 1.0 (FIG. 19A) and an MOI of 0.1 (FIG. 19B). Cells were washed three times with L-15 medium, and 5.5 ml of fresh L-15 supplied with 10% of standard or CABO-Sil-treated FBS were added to the flask. Cells were maintained at 28° C. At the indicated times post-infection, 0.5 ml of medium was removed and stored at 80° C. for later titration on Vero cells. Viral titers are estimated as average Log₁₀TCID₅₀/m±standard deviation of two independent experiments. hpi, hours post-infection.

FIGS. 20A-20C show the map and sequences for pCHIk-LR 5′GFP. FIG. 20A shows the map for plasmid CHIK-LR 5′GFP. FIG. 20B lists the primers used in the generation of this plasmid. FIG. 20C shows the sequence of this plasmid.

FIGS. 21A-21B show the map and sequences for pCHIK-LR 3′GFP. FIG. 21A shows the map for plasmid CHIK-LR 3′GFP. FIG. 21B shows the sequence of this plasmid.

FIG. 22 shows the sequences of this plasmid pChik-41855 ic.

FIGS. 23A-23C show the map and sequences for pCHIK-LR ic 1. FIG. 23A shows the map for plasmid pCHIK-LR ic 1. FIG. 23B lists the primers used in the generation of this plasmid. FIG. 23C shows the sequence of this plasmid.

FIGS. 24A-24C show the map and sequences for Chik 41855-5′GFP. FIG. 24A shows the map for plasmid Chik 41855-5′GFP. FIG. 24B lists the primers used in the generation of this plasmid. FIG. 24C shows the sequence of this plasmid.

### DETAILED DESCRIPTION OF THE INVENTION

The present invention used the alphavirus chikungunya strains (37997 and other isolates including those from LaReunion such as LR2006 isolate) to deliver nucleotide sequences of interest in vitro and in vivo. The nucleotide sequence of this strain was determined and inserted into a cDNA plasmid to produce various infectious clones of the chikungunya virus. Inserted nucleotides were expressed from a second subgenomic promoter located either 3′ or 5′ end to the structural genes of a full-length infectious alphavirus particle or from a helper plasmid containing all or part of the structural genes in conjunction with a separate plasmid containing the nonstructural genes. The nucleotides of interest were expressed from a subgenomic promoter located either on a helper plasmid or the plasmid containing the nonstructural genes in the non-full length clones. Viruses derived from the full length clones were infectious and able to replicate whereas viruses derived from non-structural/helper construct were infectious but defective in their replication. Additionally, following in vitro transcription of the linearized plasmid and electroporation of the RNA into cells, viruses were able to infect cells in vitro and mosquitoes, ticks and vertebrate in vivo.

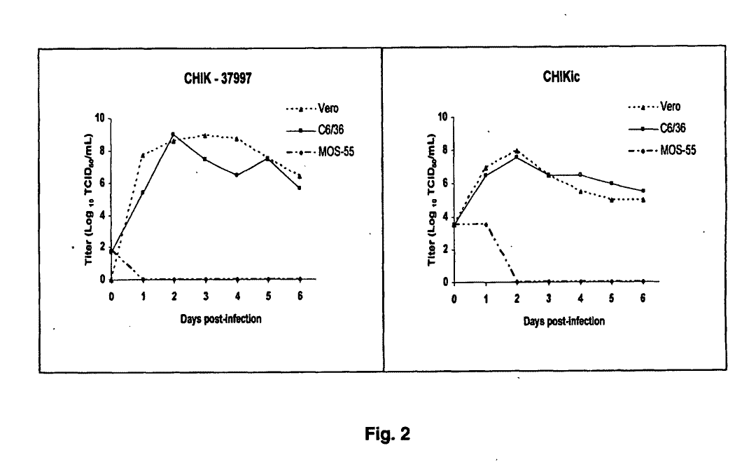

A previous study that compared the growth of chikungunya virus (37997) on Vero, C6/36, and Mos-55 cells had shown that the chikungunya virus (37997) was able to replicate in Vero and C6/36 cell lines but unable to replicate in the Moss-55 cell line (Vanlandingham et al. 2005). Neither chikungunya virus (37997) nor the virus from the infectious clone that did not contain EGFP (CHIKV) in the present invention, grew in the MOS-55 cells (FIG. 2) indicating that the in vitro phenotype of the chikungunya virus produced from the infectious clone had been retained and was similar to that of the parental virus. Furthermore, the infection rates of these two viruses were also retained in vivo following oral infection of Ae. aegypti mosquitoes (Table 2). The chikungunya virus 37997 infected 100% of the mosquitoes examined at all time points p.i. These results were similar to the virus derived from the infectious clone which infected 100% of the mosquitoes on all time points p.i. except day 3 p.i. The average whole body mosquito titer of both viruses on day 14 p.i. was 5.0 log₁₀ TCID₅₀/mL. These results and the infection data with LR-2006-based infectious clones indicated that these infectious clones would be useful for the study of chikungunya virus in Ae. aegypti and Ae. albopictus.

TABLE 2
Infection rates of CHIKV (37997) and virus derived from pCHIKic
(CHIKV), 5′ pCHIKic EGFP (5′ CHIKV EGFP) and
3′ pCHIKic EGFP (3′ CHIKV EGFP) in Ae. aegypti.

| Virus Titer¹ | Day p.i. | Titer ± S.D. | Infected/Total |
|---|---|---|---|
| CHIKV | 0 | 6.7 ± 0.3 | 3/3 (100) |
| 37997 | 1 | 5.9 ± 0.8 | 8/8 (100) |
| | 2 | 4.5 ± 0.0 | 5/5 (100) |
| | 3 | 4.8 ± 0.2 | 5/5 (100) |
| | 7 | 6.8 ± 0.6 | 8/8 (100) |
| | 14 | 5.0 ± 0.4 | 7/7 (100) |
| CHIKV | 0 | 6.5 ± 0.0 | 3/3 (100) |
| | 1 | 6.2 ± 0.5 | 8/8 (100) |
| | 2 | 5.9 ± 0.5 | 8/8 (100) |
| | 3 | 4.5 ± 0.9 | 6/8 (75) |
| | 7 | 7.0 ± 2.2 | 8/8 (100) |
| | 14 | 5.0 ± 0.4 | 7/7 (100) |
| 5′ CHIKV | 0 | 6.4 ± 0.5 | 3/3 (100) |
| EGFP | 1 | 5.0 ± 0.4 | 5/8 (63) |
| | 2 | 2.4 ± 1.2 | 4/8 (50) |
| | 3 | 2.7 ± 0.1 | 3/6 (50) |
| | 7 | 5.6 ± 0.87 | 6/6 (100) |
| | 14 | 4.2 ± 0.26 | 8/8 (100) |
| 3′ CHIKV | 0 | 6.1 ± 0.3 | 3/3 (100) |
| EGFP | 1 | 4.8 ± 0.9 | 8/8 (100) |
| | 2 | 4.4 ± 0.6 | 8/8 (100) |
| | 3 | 5.1 ± 0.9 | 8/8 (100) |
| | 7 | 5.0 ± 1.3 | 8/8 (100) |
| | 14 | 5.2 ± 0.7 | 8/8 (100) |

¹Virus titers for blood meals: CHIKV 37997 - 7.95 log₁₀ TCID₅₀/mL, virus from pCHIKic - 7.95 log₁₀ TCID₅₀/mL, 5′ CHIKV EGFP - 7.52 log₁₀TCID₅₀/mL, 3′CHIKV EGFP - 7.52 log₁₀TCID₅₀/mL.

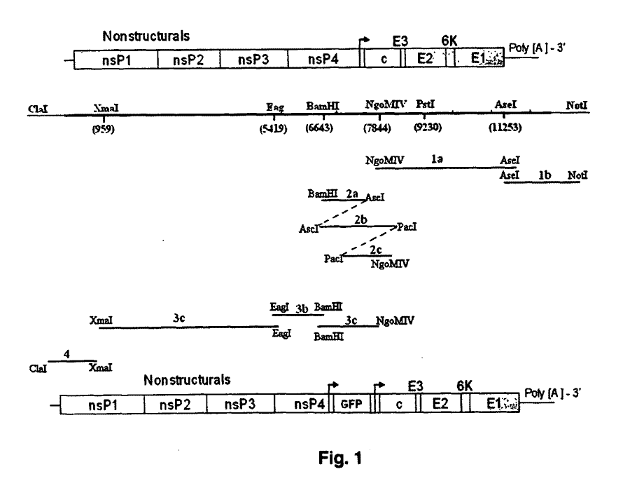

Additionally, the two viruses that expressed the reporter gene, EGFP were compared in Ae. aegypti mosquitoes. These viruses differed in the placement of the EGFP sequence within the viral genome. Previous studies had indicated that the placement of the reporter gene at either the 5′ or the 3′ position within various alphaviruses produced differences in expression levels of the reporter gene and in the stability of the construct (Higgs et al. 1995). In the 5′ pCHIKic EGFP, the EGFP was placed downstream of the non-structural genes and a RNA subgenomic promoter. The EGFP was followed by an additional internal RNA subgenomic promoter sequence and the viral structural genes (FIG. 1). The 5′ position had been shown to be more stable in two SINV expression systems (ME2 5′2J/GFP and TE/5′2J/GFP) following several passages in cell culture. The genes encoding GFP placed at the 5′ position expressed GFP in more than 90% of the cells following five passages.

In the 3′ pCHIKic EGFP construct, the EGFP was expressed from an additional RNA subgenomic promoter which was located at the extreme 3′ end of the structural genes of the virus. Studies using various SIN expression systems had indicated that the 3′ construction was unstable after multiple passages in cell culture. This instability was characterized by the ability to detect viral antigen in the absence of GFP expression (Higgs et al. 1999).

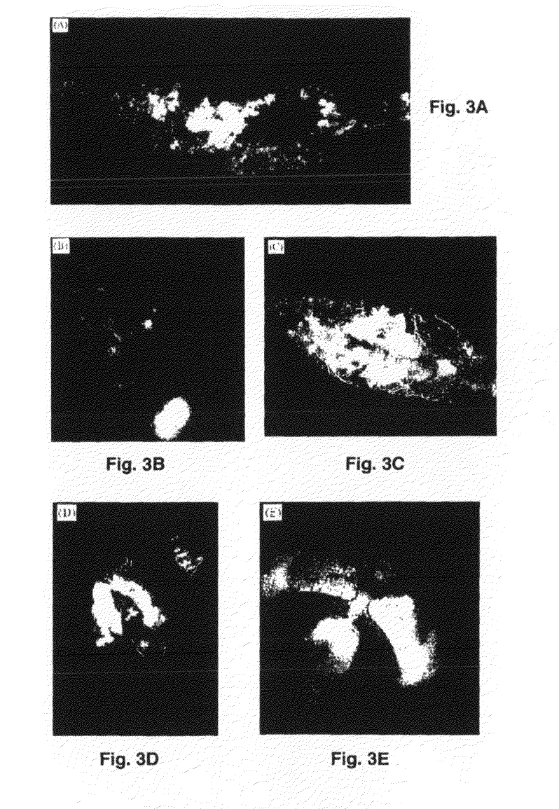

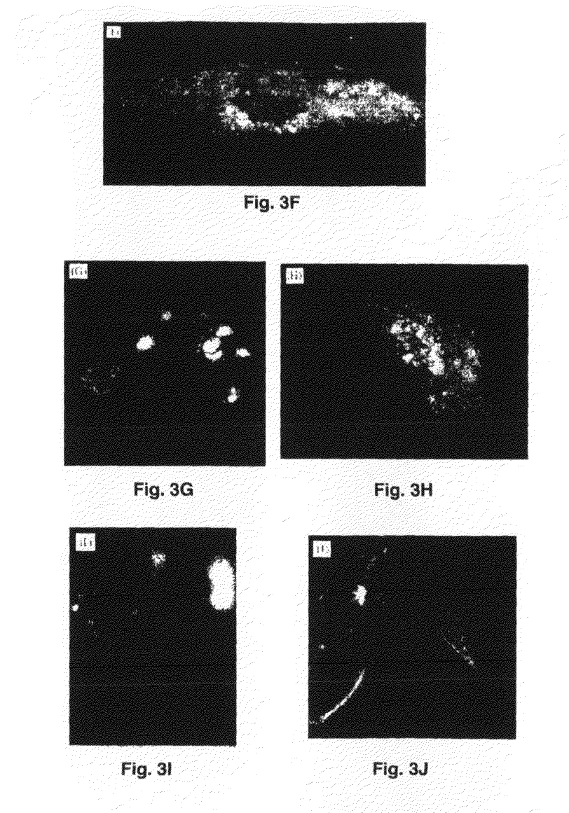

Ae. aegypti mosquitoes infected with either the 5′ or 3′ chikungunya virus EGFP were analyzed by IFA and EGFP expression in the midguts and salivary glands on days 7 and 14 p.i. Nervous tissue was also examined on day 14 p.i. by analysis of EGFP expression in the eyes (FIG. 3). These tissues and time points were selected based on previous experiments with O'nyong-nyong virus (ONNV) and chikungunya virus (Vanlandingham et al., 2005). The tissue tropisms of chikungunya virus EGFP differed from those observed for sindbis virus at similar time points (Foy et al., 2004; Pierro, et al., 2003; Reyms-Keller et al., 1995) being less focal in the midgut at early time points and more intense in infected tissues at late time points.

EGFP was expressed in a higher percent of the salivary glands on day 14 p.i. for 5′CHIKV EGFP when compared to 3′CHIKV EGFP (Table 3). The intensity of EGFP expression was greater for 3′CHIKV EGFP on day 14 p.i. (FIG. 3). The 3′ CHIKV EGFP disseminated in 100% of the mosquito salivary glands examined by IFA and 70% of the mosquito salivary glands and eyes when examined by EGFP expression (Table 3). The finding that virus disseminated at a higher level than the expression of EGFP had been demonstrated for other alphavirus expression systems which used the 3′ construction (Olson et al. 2000).

TABLE 3
Infection and dissemination rates based on antigen detection by IFA
and by visualization of EGFP for the CHIKV 37997 and viruses
derived from the pCHIKic, 5′pCHIKic EGFP and 3′pCHIKic
EGFP in Ae. Aegypti mosquitoes.

| Virus | Day | | | | |
|---|---|---|---|---|---|
| strain | p.i | Infected (IFA)¹ | | | |
| CHIKV | 7 | 10/10 (100) | 9/10 (100) | na³ | |
| 37997 | 14 | 10/10 (100) | 10/10 (100) | na | na |
| CHIKV | 7 | 9/10 (90) | 9/10 (90) | na | na |
| | 14 | 7/10 (70) | 5/10 (50) | na | na |
| 5′ CHIKV | 7 | 10/10 (100) | 8/10 (80) | 10/10 (100) | 7/10 (70) |
| EGFP | 14 | 10/10 (100) | 10/10 (100) | 10/10 (100) | 9/10 (90) |
| 3′ CHIKV | 7 | 5/5 (100) | 1/5 (20) | 10/10 (100) | 4/10 (40) |
| EGFP | 14 | 5/5 (100) | 5/5 (100) | 10/10 (100) | 7/10 (70) |

¹Virus titers of blood meals, analyzed by IFA: CHIKV 37997 - 7.95 log₁₀ TCID₅₀/mL, viruses produced from pCHIKic - 7.95 log₁₀ TCID₅₀/mL, 5′ pCHIKic EGFP - 7.52 log₁₀ TCID₅₀/mL, 3′ pCHIKic EGFP - 7.52 log₁₀ TCID₅₀/mL.
²Virus titers of blood meal titers, analyzed by EGFP: 5′ pCHIKic EGFP - 7.95 nd⁴, 3′ pCHIKic EGFP - 7.95 log₁₀ TCID₅₀/mL,
³na = not applicable

Based on the results obtained, it is contemplated that the high level of infection and efficient dissemination of these three chikungunya virus infectious clones will enable studies of virus tropisms in an epidemiologically important vector with a naturally infectious virus. Additionally, the 5′ and 3′CHIKV constructs will provide additional tools for gene expression and knockout studies, for example RNAi, in Ae. aegypti mosquitoes. Furthermore, by increasing the repertoire of alphaviral infectious clones, chimeric viruses with specific gene or amino acid substitutions can be produced that will help in the identification of the molecular determinants of the viral infection process in mosquitoes. Using the methodology described herein, the present invention has generated clones of CHIKV 41855 using the full-length and replicon system of this virus.

The present invention further examined the infectious clones of chikungunya virus (La Reunion isolate) for vector competence. Phylogenetic analysis of the sequences of CHIKV isolates obtained during the current outbreak indicate that these sequences form a homogeneous Glade within the Central/EastAfrican genotype of CHIKV and are most closely related to the isolates from Central Africa (Parola et al. 2006, Schuffenecker et al. 2006, Powers et al. 2000). Recently, the development and characterization of infectious clones of CHIKV strain 37997 (Vanlandingham et al. 2005a) which belongs to the West African genotype of CHIKV and is only distantly related to the strains from La Réunion Island was reported. This may restrict the application of the CHIKV strain 37997 clones due to differences from the virus of interest in the islands in the Indian Ocean. It was therefore considered important to construct infectious clones of a La Réunion isolate of CHIKV to further the understanding of the molecular mechanisms that have contributed to the unprecedented magnitude of the La Réunion outbreak.

CHIKV LR2006 OPY strain was fully sequenced during the construction of the infectious clone. The coding sequence was found to differ at six positions with respect to the sequence of LR2006 OPY 1 originally deposited in GenBank (DQ443544). This deposited sequence has been amended to take the differences into account.

The specific infectivity of the RNA produced from CHIK-LR is was 8×10⁵ pfu/μg of RNA. This is close to the upper limits of detection and is similar to the specific infectivity of other well-characterized alphavirus clones (Liljestrom et al. 1991, Rice et al. 1987). The growth kinetics of virus produced from this infectious clone were similar to the original parental virus in both cell culture (FIGS. 11A-11B) and in Ae. aegypt and Ae. albopictus mosquitoes (Table 5). Infection rates of both viruses were near 100% in Ae. albopictus mosquitoes, whereas they reached only 60-78% in Ae. aegypti (Table 5). This is similar to previously reported infection rates of CHIKV infection in mosquitoes (Turell et al. 1992). Interestingly, experiments using CHIKV strain 37997 demonstrated the infection rate to be 100% for Ae. aegypti (Vanlandingham et al. 2005a, 2006). One possible explanation for this discrepancy is that the genetic differences between these two CHIKV strains influence the infectivity for Ae. aegypti. Differences may also be due to a change of protocol for the production of the infectious blood meal, i.e. from Vero to C6/36 cells. Thus, it has been shown that the biological properties of a virus can vary depending on the origin of the infectious particles, possibly due to differences in the glycosylation pattern (Klimstra et al. 2003, Hsieh et al. 1984).

Two additional double sub-genomic infectious clones, CHIKV-LR 5 GFP and CHIKV-LR3 GFP, were developed and characterized in cell culture (FIGS. 11A-11B), in Ae. aegypti and Ae. albopictus (FIGS. 13A-13E and Table 7). The specific infectivity of the RNA produced from these clones was similar to that obtained from virus derived from CHIKV-LR ic indicating that the introduced genetic changes were not lethal for the virus (Table 4). The level of GFP expressed by BHK-21 cells transfected with CHIKV-LR 3 GFP RNA was much higher than GFP expressed using the CHIKV-LR 5 GFP construct (FIGS. 13A-13E). Higher levels of GFP expression in alphavirus infectious clones constructed using the 3′ configuration have been reported (Bredenbeek et al. 1992, Pugachev et al. 1995, Vanlandingham et al. 2005a). The growth of both viruses derived from the 3′ or 5′ infectious clones in Vero and C6/36 cells was attenuated as compared with that of CHIKV-LR and virus produced from CHIKV-LR ic indicating that these viruses might be unstable and can lose the ability to express GFP following serial passage in cell culture (Table 6 and FIGS. 12A-12B). The phenomenon of instability of double subgenomic (ds) viruses has been described for several alphaviruses including Sindbis virus and o'nyong nyong virus (Higgs et al. 1995, Pugachev et al. 1995, Pierro et al. 2003, Brault et al. 2004). Generally, 5 constructs have been shown to be more stable than 3 constructs. It was also observed that during serial passage, GFP expression from both ds viruses decreased with passage number and that CHIKV-LR 5 GFP was more stable than CHIKV-LR 3 GFP in both cell lines (Table 6). This is most likely due to the placement of the second subgenomic promoter and the GOI in the center of the genome which leads to a decrease in the probability of recombination events around this region.

Interestingly, since the multiplicity of infection was similar between CHIKV-LR 5 GFP and CHIKV-LR 3 GFP, the data presented herein also indicate that the CHIKV-LR 3 GFP is more stable in Vero cells than in C6/36 cells (Table 6). It is possible that the region of the viral genome where the second subgenomic promoter was introduced contains sites for binding of some cellular proteins that are important for viral replication. Kuhn et al. (1990) demonstrated that deletion of parts of the 3 non-coding region (NCR) of Sindbis virus had a much stronger effect on virus replication in mosquito cells as compared with CEF (chicken embryo fibroblast) cells (Kuhn et al. 1990).

Infection rates of Ae. aegypti and Ae. albopictus mosquitoes orally infected with virus derived from CHIKV-LR 5 GFP were similar to those observed for CHIKV-LR and virus derived from CHIK-LR ic, indicating that the introduction of the GFP gene into the viral genome does not significantly impair viral phenotype (Tables 5 and 7). Analysis of the GFP expression in mosquito tissues revealed that virus derived from CHIKV-LR 5 GFP can escape the midgut barrier and disseminate to the salivary gland of both mosquito species (FIGS. 13A-13E). Interestingly, GFP expression was different in Ae. aegypti, where GFP was localized primarily in the posterior parts of the midgut, and in Ae. albopictus, where GFP was found throughout the midgut epithelial cells (FIG. 13A-13E). This indicates that cell permissiveness during initial viral infection is not homogeneously distributed in the Ae. aegypti midgut. Similar results have been reported for the MRE 1001 strain of Sindbis virus (Pierro et al. 2003).

The pattern of GFP expression in Ae. aegypti and Ae. albopictus mosquitoes infected with virus derived from CHIKV-LR 3 GFP was markedly different compared with virus derived from CHIKV-LR 5 GFP. Virus derived from CHIKV-LR 3 GFP was able to infect Ae. albopictus midguts. However, no GFP fluorescence was observed in the mosquito salivary glands (FIGS. 13A-13E). This most likely reflects the instability of the virus; only viruses that lacked GFP were able to disseminate to the salivary glands. Thus, when mosquito bodies were titrated, GFP fluorescence was not detected in every dilution well that showed cytopathic effects. Similar results have suggested instability of another alphavirus infectious clone, MRE/3 2J/GFP, in Ae. aegypti (Pierro et al. 2003).

CHIKV-LR 3 GFP was unable to infect Ae. aegypti by the oral route of infection (Table 7), although in a previous study (Vanlandingham et al. 2005a), 3′CHIKV EGFP virus was shown to be infectious for this species of mosquito. It is possible that the LR2006 OPY strain of CHIKV might have sites recognized by mosquito proteins that are important for viral replication in the region where the GFP gene was introduced. Since the 37997 and LR2006 OPY strains have only 80-85% sequence identity among ORFs and even less in the 3′NCR, it is conceivable that 3′CHIKV EGFP would behave differently in the same mosquito species. Nevertheless, the development of three new CHIKV infectious clones based on the epidemic LR2006 OPY strain obtained from La Réunion will be invaluable tools to study molecular determinants of infection in mosquitoes and pathogenicity in vertebrate hosts, and may provide data for the development of novel methods to control this reemerging virus.

The CHIKV outbreak in Reunion is unique because it is the first well-documented report of an alphavirus outbreak for which Ae. albopictus was the main vector. Interestingly, this was also the first Chikungunya epidemic during which fatal infections were reported. The data presented herein clearly indicate that an E1-A226V mutation in CHIKV results in increased fitness of CHIKV in Ae. albopictus mosquitoes with respect to midgut infectivity, dissemination to the salivary glands, and transmission to a vertebrate species. These data demonstrate that a single E1-A226V mutation is sufficient to dramatically increase the ability of different strains of CHIKV to infect Ae. albopictus mosquitoes and that this substitution requires no additional adaptive mutations to gain intermolecular compatibility. These complimentary experimental data demonstrate that a single mutation is sufficient to modify viral infectivity for a specific vector species and as a consequence, can fuel an epidemic in a region that lacks the typical vector. These observations provide the basis for an explanation of the observed rapid shift among CHIKV genotypes to viruses containing the E1-A226V mutation during the Reunion outbreak (Schuffenecker et al., 2006).

Interestingly, the data presented herein and data from previous studies (Turell et al., 1992; Tesh et al., 1976) indicate that prior to acquiring the E1 A226V mutation, CHIKV is capable of producing high enough viremia in humans to efficiently infect Ae. albopictus mosquitoes. One explanation of the evolutionary force which allowed CHIKV to be selected so rapidly into a CHIKV strain which is adapted to Ae. albopictus, is that the increased infectivity (lower OID₅₀) of CHIKV E1-A226V mutants for Ae. albopictus means that the human viremic thresholds required for Ae. albopictus infection would likely occur earlier and be sustained for longer. Several recent studies indicate that during the course of human viremia, which last up to 6 days, CHIKV loads can reach up to 3.3×10⁹ RNA copies per ml of the blood (Parola et al., 2006, Carletti et al., 2007), which corresponds to 6-7 Log₁₀TCID₅₀/ml (Carletti et al., 2007). Earlier studies that utilized a suckling mouse brain titration protocol, which is more sensitive than titration on Vero cells, also found that human viremia often exceeded 6 Log₁₀SMICLD₅₀/0.02 ml (Carey et al., 1969). Based on viremia studies in rhesus monkeys that can develop up to 7.5 Log/ml if assayed by suckling mice brain titration (Paul and Singh, 1968) and a maximum viremia of only 5.5 Log10/ml based on Vero cell titration (Binn et al., 1967), it may be possible that viremias in humans would correlate to 6-7 Log₁₀TCID₅₀/ml. From these data, the maximum virus load which can be achieved in human blood may be calculated to be 1-2 Log₁₀TCID₅₀/ml higher than the Log₁₀OID₅₀/ml for E1-226A viruses but 3-4 Log₁₀TCID₅₀/ml higher than the Log₁₀OID₅₀/ml for E1-226V viruses.

During the course of viremia there should therefore be a substantial time frame in which CHIKV blood load is high enough for E1-226V viruses to infect Ae. albopictus but below the threshold for infection with E1-226A viruses. This increased opportunity for Ae. albopictus infection, would perpetuate the selection and transmission of the mutant virus.

During transmission competition assays, only E1-226V virus was transmitted to suckling mice by Ae. albopictus, although in these experiments, titers of E1-226V and E1-226A viruses were of a high enough magnitude to allow both of these viruses to efficiently infect this mosquitoes species. This indicates that there are additional mechanisms that could ensure evolutional success of the E1-A226V viruses transmitted by Ae. albopictus. It is possible that one of these mechanisms is associated with more efficient dissemination of the E1-226V as compared with E1-226A viruses. This could shorten the extrinsic incubation period (EIP)—the time from mosquito infection to transmission—and could have contributed to the evolutionary success of CHIKV during the Reunion outbreak because vectors infected with the LR-226V virus would transmit it more quickly than those infected with LR-226A viruses. Additionally, with relatively short-lived vectors such as mosquitoes (Christophers, 1960), longer EIPs reduce transmission efficiency simply because fewer mosquitoes survive long enough to transmit the virus.

The present invention does not provide data to determine if dissemination efficiency of the E1-226V viruses into the salivary glands is a consequence of more efficient midgut infectivity or if these two phenomena are independent. In this regard, it will be of particular interest to investigate the effect of the E1-A226V mutation on CHIKV transmission by orally or intrathoracically infected Ae. albopictus mosquitoes.

Although the CHIKV E1-A226V mutation gives a selective advantage in Ae. albopictus, there was not a corresponding advantage in Ae. aegypti. The OID₅₀ and midgut competition assay data indicate that E1-226V viruses were slightly less infectious for midgut cells of Ae. aegypti mosquitoes (FIGS. 14C, 14D, and 15C; Table 10). Additionally, in contrast to Ae. albopictus, E1-226V viruses do not have a detectable advantage for dissemination into salivary glands and heads of Ae. aegypti. In transmission competition experiments from Ae aegypti to suckling mice, E1-226V conferred a slight competitive advantage over E1-226A (FIG. 18C). However, five out of six mice exposed to CHIKV infected Ae aegypti had equivalent amounts of both E1-226A and E1-226V viral RNAs. These results are markedly different compared to the results obtained in similar experiments using Ae. albopictus mosquitoes and further support the hypothesis that this E1-A226V was specifically selected as a result of adaptation of CHIKV to Ae. albopictus mosquitoes. To explain the small fitness advantage associated with the E1-A226V mutation which was observed in transmission experiments, it is hypothesized that, similarly to Ae. albopictus, E1-226A and E1-226V viruses colonize different Ae. aegypti organs at different efficiencies. E1-226A appears to colonize midgut cells of Ae aegypti better than E1-226V viruses; however, following dissemination into salivary glands, the E1-226V virus gains an advantage for transmission to vertebrates.

The E1-A226V mutation was found to have a slightly negative effect on infectivity, a negligible effect on dissemination, but a slight positive effect on transmissibility of CHIKV by Ae. aegypti in the competition experiment. It is contemplated that these small (as compared with Ae. albopictus) differences associated with the E1-A226V mutation would not be sufficient to have a significant effect on the evolution of CHIKV transmitted by Ae. aegypti and would not result in accumulation of this mutation in the regions where Ae. aegypti serves as a primary vector for CHIKV. This may explain the lack of emergence of the E1-226V genotype in previous outbreaks and the predominance of E1-226A viruses during the 2006 CHIKV epidemic in India, in which Ae. aegypti is considered to be the main vector species. Adaptation of African strains of CHIKV from forest dwelling mosquitoes species to Ae. aegypti has never been shown to be associated with any particular mutations, therefore it is believed that the same negative impact of E1-A226V would be seen in African mosquito vectors which were responsible for transmission of CHIKV strains ancestral to Reunion isolates.

The data presented herein does not exclude the possibility that the E1-A226V mutation might have a negative effect on the evolution of CHIKV transmitted by Ae. aegypti. Since our dissemination and transmission studies were performed using blood meal titers that were 1-2 Log₁₀TCID₅₀/ml higher than Log₁₀OID₅₀/ml values, it may be possible that the negative effect of decreased midgut infectivity of E1-A226V on virus trans-missibility would be almost completely missed, simply because, under this condition, almost 100% of mosquitoes could become infected. In general, CHIKV requires significantly higher blood meal titers for infection of Ae. aegypti compared to Ae. albopictus (Turell et al., 1992; Tesh et al., 1976) (Tables 9 and 10), which suggests that the slight decrease in midgut infectivity of E1-226V viruses would have a more profound effect on the evolution of CHIKV transmitted by Ae. aegypti, compared to the effect of a small advantage in the ability to compete with E1-226A viruses for transmission to suckling mice. Therefore, if the E1-A226V mutation occurred in CHIKV transmitted by Ae. aegypti, it would have a weak negative effect on viral fitness and would most likely not be preferentially selected. Additional experiments are required to evaluate this hypothesis.

Available data cannot exclude the possibility that E1-226A viruses may have an unknown beneficial effect on the fitness of CHIKV in vertebrate hosts over E1-226V viruses, and that the minor negative effect of E1-226A observed in transmission experiments by Ae. aegypti can be compensated for by more efficient viral replication in the vertebrate host, leading to an overall more efficient adaptation to the transmission cycle. However, comparison of the different effects of A or V residues at position E1-226 on CHIKV infectivity for, and transmission by Ae. aegypti and Ae. albopictus mosquitoes clearly suggests that polymorphisms at this position may determine the host range of the alphaviruses and may play an important role in adaptation of the viruses to a particular mosquito vector.

An interesting observation, which should be studied in more detail, was that adaptation of CHIKV to Ae. albopictus mosquitoes coincided with the acquisition of CHIKV dependence on cholesterol in the target membrane. It has been previously shown that various mutations in the same region of the E1 protein of SFV and Sindbis virus can modulate the cholesterol dependence of these viruses (Vashishtha et al., 1998; Lu et al., 1999) and that SFV independence from cholesterol coincides with more rapid growth of the virus in Ae. albopictus (Ahn et al., 1999). Although there is an apparent association, it is currently unknown if cholesterol dependence of alphaviruses is directly responsible for modulation of fitness of alphaviruses in mosquito vectors. A possible explanation for the opposite effects of the cholesterol-dependent phenotype of SFV and CHIKV on fitness in Ae. albopictus may reflect the use of different techniques for mosquito infection. In the present invention, mosquitoes were orally infected via cholesterol rich blood meals, whereas in the previous study SFV was intrathoracically inoculated into the mosquito (Ahn et al., 1999). It is also possible that cholesterol-dependent and -independent viruses would replicate differently in different mosquito organs. As such, the data presented herein indicate that more efficient colonization of Ae. albopictus midgut cells by cholesterol-dependent LR-ApaI-226V is followed by relatively more rapid growth of cholesterol-independent LR 226A virus in mosquito bodies between 3 and 5 dpi (FIG. 17B). Three to 5 dpi coincides with virus escape from the mosquito midgut.

Alignment of amino acid sequences that constitute the ij loop of E1 protein from different members of the alphaviruses genus revealed that position E1-226 is not conserved (Vashishtha et al., 1998 and data not shown) and can vary even between different strains of the same virus. In this regard, it would be reasonable to determine the cholesterol requirement of other clinically important alphaviruses, especially Venezuelanequine encephalitis virus (VEEV) and eastern equine encephalitis virus (EEEV), which show significant intra-strain variation at position E1-226 among natural isolates of these viruses, and determine mutations which can modulate their cholesterol dependence. In recent studies by Kolokoltsov et al., it was suggested that VEEV, a New world alphavirus, might be cholesterol independent, although the use of Vero cells instead of C6/36 cells, and the use of different protocols for cell membrane cholesterol depletion, make it difficult to compare the results of this study with our findings. Also it would be of interest to determine possible relationships between mutations which modulate cholesterol dependence of alphaviruses other than CHIKV and on their infectivity for Ae. aegypti and Ae. albopictus mosquitoes and perhaps other epidemiologically important mosquito vectors.

The molecular mechanisms responsible for the association between host range and cholesterol dependence of CHIKV are unknown (Kielian and Rey, 2006). It has been proposed that upon exposure to low pH, the E1 protein of cholesterol-dependent viruses senses the target membrane lipid composition and goes through a cholesterol-dependent priming recognition reaction (Chatterjee et al., 2000), which is not required for cholesterol-independent viruses. It is possible that CHIKV infects Ae. aegypti and Ae. albopictus midgut cells using different endocytic pathways, which targets virus to cellular compartments with different lipid contents in which fusion occurs. Specific lipids such as cholesterol may differentially affect fusion of cholesteroldependent and cholesterol-independent CHIKV strains in these compartments and therefore define the outcome of infection. Although the observations discussed herein are suggestive, more comprehensive studies should be completed to determine the exact molecular mechanisms responsible for penetration of E1-226A and E1-226V viruses into Ae. aegypti and Ae. albopictus cells.

Although previous laboratory studies have demonstrated susceptibility of Ae. albopictus to CHIKV infection (Turell et al., 1992; Tesh et al., 1976), the data herein demonstrate that the E1-A226V mutation promoted infection and accelerated dissemination of CHIKV in Ae. albopictus mosquitoes and conferred a selective advantage over infection of Ae. aegypti. Whilst the mutation did not increase the maximum viral titer attainable in the mosquitoes, the synergistic effects of increased infectivity and faster dissemination of the E1-A226V virus in Ae. albopictus would accelerate virus transmission to a nai\ddot{}ve human population which would have contributed to initiating and sustaining the 2005-2006 CHIKV epidemic on Reunion island. That a single amino acid change can act through multiple phenotypic effects to create an epidemic situation has implications for other arthropod-transmitted viruses and the evolution of human infectious diseases (Wolfe et al., 2007).

The present invention is directed to an expression vector comprising a DNA sequence encoding a full length chikungunya virus comprising non-structural protein genes and structural protein genes of the CHIKV. The DNA sequence encoding the non-structural protein genes may be inserted in one plasmid and the DNA sequence encoding the structural protein genes may be inserted in a second plasmid. Alternatively, the DNA sequence encoding the non-structural protein genes may be inserted in one plasmid, the DNA sequence encoding the capsid structural protein may be inserted in a second plasmid and the DNA sequence encoding the rest of the structural genes may be inserted in a third plasmid. Additionally, the structural protein gene(s) in the expression vector may comprises a single amino acid substitution effective to increase infectivity of an chikungunya virus infectious clone in a mosquito. The amino acid substitution may include but is not limited to A226V mutation in E1 protein of the chikungunya virus.

Examples of chikungunya virus strains from which such a DNA sequence is derived is not limited to but includes 37997, strain Nagpur (India) 653496, strain S27-African prototype, strain Ross or LR2006 isolates from LaReunion. The expression vector described herein further comprises a heterologous gene, a knock-out gene, an over-expressing gene or an immunogenic sequence. Examples of such genes are known in the art. Therefore based on the information disclosed in present invention, one skilled in the art can easily construct expression vectors expressing these genes.

The present invention is further directed to a host cell comprising and expressing the vector comprising a DNA sequence encoding a full length chikungunya virus comprising non-structural protein genes and structural protein genes of the CHIKV. Additionally, the present invention is also directed to an infectious clone comprising the DNA sequence encoding a full length chikungunya virus comprising non-structural protein genes and structural protein genes of the CHIKV. Such a clone encodes an attenuated chikungunya virus.

Furthermore, the present invention is also directed to a pharmaceutical composition comprising the attenuated chikungunya virus encoded the infectious clone described herein, where the structural protein gene(s) in the clone does not comprise the amino acid substitution, DNA sequence encoding a protein of interest expressed by the clone and a pharmaceutically acceptable carrier. The DNA sequence encoding protein of interest is not limited to but includes DNA sequence of a heterologous gene, an overexpressed gene, a knockout/knock down genes or an immunogenic peptide.

The present invention is further directed to an immunogenic composition comprising a live attenuated chikungunya virus encoded by the clone described herein, where the structural protein gene(s) in the clone does not comprise the amino acid substitution and a DNA sequence encoding an immunogenic peptide expressed by the clone. The present invention is further yet directed to an immunogenic composition comprising an attenuated chikungunya virus encoded by the clone described herein, where the structural protein gene(s) in the clone does not comprise the amino acid substitution, where the attenuated CHIKV is inactivated and a DNA sequence encoding an immunogenic peptide expressed by the clone.

The present invention is also directed to a method of evaluating function of a gene in an organism, comprising expressing the gene or knocking out the gene using the above-discussed infectious clone, and determining the effect of the over-expressing or knocking out the gene in the organism, thereby evaluating the function of the gene in the organism.

The present invention is further directed to a method of inducing protective immune response in a subject, comprising: administering pharmaceutically effective amounts of an immunogenic composition comprising either a live attenuated chikungunya virus or an inactivated chikungunya virus and an immunogenic peptide discussed supra, thereby inducing a protective immune response in the subject. Generally, the subject is a human or a non-human primate.

Alternatively, the present invention is also directed to an expression vector comprising a DNA sequence encoding a full length chikungunya virus comprising non-structural protein genes and structural protein genes of the CHIKV and an additional subgenomic promoter. The DNA sequence encoding the non-structural protein genes of the CHIKV may be inserted in one plasmid and the DNA sequences encoding the structural protein genes and the subgenomic promoter may be inserted in a second plasmid. Alternatively, the DNA sequence encoding the non-structural protein genes may be inserted in one plasmid, the DNA sequence encoding capsid structural protein gene may be inserted in a second plasmid and the DNA sequence encoding the rest of the structural protein genes and the sub-genomic promoter may be inserted in a third plasmid. The additional subgenomic promoter is placed either 3′ or 5′ to the structural protein genes. Additionally, the structural protein gene(s) in the expression vector may comprises a single amino acid substitution effective to increase infectivity of an chikungunya virus infectious clone in a mosquito. The amino acid substitution may include but is not limited to A226V mutation in E1 protein of the chikungunya virus.

The strain from which the CHIKV DNA sequences are derived and the examples of the genes that can be expressed using this expression vector is as discussed supra. The present invention is directed to a host cell comprising and expressing a vector comprising a DNA sequence encoding a full length chikungunya virus comprising non-structural protein genes and structural protein genes of the CHIKV and an additional subgenomic promoter. Additionally, the present invention is also directed to an infectious clone comprising the DNA sequence encoding a full length chikungunya virus comprising non-structural protein genes and structural protein genes of the CHIKV and an additional subgenomic promoter. Such a clone encodes an attenuated chikungunya virus. Attenuation is encoded in the structural gene sequences.

Furthermore, the present invention is also directed to a pharmaceutical composition comprising the attenuated chikungunya virus encoded by the infectious clone described herein, where the structural protein gene(s) in the clone does not comprise the amino acid substitution, a DNA sequence encoding a protein of interest expressed by the clone and a pharmaceutically acceptable carrier. The DNA sequence encoding protein of interest is not limited to but includes DNA sequence of a heterologous gene, an overexpressed gene, a knockout/knock down gene or an immunogenic peptide.

The present invention is still further directed to an immunogenic composition comprising a live attenuated chikungunya virus and a subgenomic promoter encoded by the clone described herein, where the structural protein gene(s) in the clone does not comprise the amino acid substitution, and a DNA sequence encoding an immunogenic peptide expressed by the clone. The present invention is further yet directed to an immunogenic composition comprising an attenuated chikungunya virus and a sub-genomic promoter encoded by the clone described herein, where the chikungunya virus is inactivated and a DNA sequence encoding an immunogenic peptide expressed by the clone.

The present invention is also directed to a method of evaluating the function of a gene in an organism, comprising expressing the gene or knocking out the gene using the above-discussed infectious clone, and determining the effect of the over-expressing or knocking out the gene in the organism, thereby evaluating the function of the gene in the organism.

The present invention is further directed to a method of inducing a protective immune response in a subject, comprising: administering pharmacologically effective amounts of an immunogenic composition comprising either a live attenuated chikungunya virus, a subgenomic promoter and an immunogenic peptide or an inactivated chikungunya virus, subgenomic promoter and an immunogenic peptide discussed supra, thereby inducing a protective immune response in the subject. Generally, the subject is a human or a non-human primate.

The present invention is also directed to a CHIKV replicon system, comprising a replicon comprising non-structural genes of the CHIKV and a marker gene and a helper system comprising structural genes of the CHIKV. Examples of marker gene may include but are not limited to a gene encoding green fluorescent protein as well as other marker genes well know to those having ordinary skill in this art. The replicon system can be generated using the structural and non-structural of the CHIKV discussed supra. The structural protein gene(s) in the replicon may comprises a single amino acid substitution effective to increase infectivity of a chikungunya virus like particle in a mosquito. The amino acid substitution may include but is not limited to A226V mutation in E1 protein of the chikungunya virus. Additionally, the present invention is also directed to a host cell, comprising and expressing the CHIKV replicon system discussed herein.

The present invention is also directed to a virus like particle comprising genes encoded by the replicon system discussed supra.

The present invention is further directed to a method of identifying sites of primary CHIKV infection in a mosquito vector, comprising: feeding the virus like protein discussed supra to the mosquito vector and detecting expression of the marker gene in the midgut and salivary gland of the mosquito vector, thereby identifying sites of primary CHIKV infection in the mosquito vector.

As used herein, the term, “a” or “an” may mean one or more. As used herein in the claim(s), when used in conjunction with the word “comprising”, the words “a” or “an” may mean one or more than one. As used herein “another” or “other” may mean at least a second or more of the same or different claim element or components thereof.

As discussed herein, the structural proteins in the expression vectors and replicon system may comprise a single amino acid substitution in one or more of the structural proteins of the chikungunya virus. This substitution would increase the infectivity of the clones derived therefrom or virus like particle in mosquito. However, since clones used to generate immunogenic or pharmaceutical compositions must not infect mosquitoes, these clones should not comprise such substitutions in the structural proteins.

The composition described herein can be administered either systemically or locally, by any method standard in the art, for example, subcutaneously, intravenously, parenterally, intraperitoneally, intradermally, intramuscularly, topically, enterally, rectally, nasally, buccally, vaginally or by inhalation spray, by drug pump or contained within transdermal patch or an implant. Dosage formulations of the composition described herein may comprise conventional non-toxic, physiologically or pharmaceutically acceptable carriers or vehicles suitable for the method of administration. The composition described herein may be administered one or more times to achieve, maintain or improve upon a therapeutic effect. It is well within the skill of an artisan to determine dosage or whether a suitable dosage of either or both of the composition comprises a single administered dose or multiple administered doses. An appropriate dosage depends on the subject's health, the induction of the desired effect, the route of administration and the formulation used.

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. Changes therein and other uses which are encompassed within the spirit of the invention as defined by the scope of the claims will occur to those skilled in the art.

### Example 1

### Viruses

The 37997 strain of CHIKV was obtained from the World Reference Center for Arboviruses at the University of Texas Medical Branch, Galveston, Tex. CHIKV was originally isolated from Ae furcifer mosquitoes from Kadougou, Senegal in 1983 and was passed once in Ae. pseudoscutellaris (AP-61) cells and twice in Vero (green monkey kidney) cells. Stock virus was produced following a single passage in Vero cells, grown at 37° C. in Leibovitz L-15 media with 10% fetal bovine serum (FBS), 100 U penicillin, and 100 g/mL streptomycin. Virus was harvested when cells showed 75% cytopathic effect (CPE) and aliquoted and stored at −80° C. for use in all experiments.

### Example 2

### RNA Extraction

RNA was generated using C6/36 cells that were inoculated with the CHIKV (37997). Virus was harvested from cell culture supernatant using the QIAamp Viral RNA Mini kit (Qiagen, Valencia, Calif.) following the manufacturer's protocol. RNA was stored at −80° C. for later use.

### Example 3

### Reverse Transcription and Sequencing

CHIKV (37997) RNA was reverse transcribed to produce cDNA using random hexanucleotide primers (Promega, Madison, Wis.) and Superscript II (Invitrogen Life Technologies) following manufactures instructions. cDNA was amplified with Taq DNA polymerase (New England BioLabs, Beverly, Mass.) with 35 cycles at 94° C., 20 sec; 55° C., 20 sec; 72° C., 2 min; final extension at 70° C. for 5 min. Amplified PCR products were analyzed by electrophoreses on 1% agarose gel and gel-purified using the QIAquick Gel Extraction Kit (Qiagen). The purified PCR products were used for direct sequencing.

### Example 4

### Sequencing of the 5′ and 3′ Ends

The 3′ terminal sequence was determined using the 3′ RACE method (Frohman 1994). The 5′ terminal sequence was determined using the 5′ RACE kit (Ambion, Austin, Tex.) following the manufacturer's instruction.

### Example 5

### Construction of Infectious Clones

Three plasmids, pCHIKic, 5′ pCHIKic EGFP, and 3′ pCHIKic EGFP, were prepared by standard PCR-based cloning methods. CHIKV DNA fragments were substituted into an alphavirus o'nyong nyong/pBluescript II SK(+) infectious clone (p5′dsONNic-Foy) which was provided by Ken E. Olson and Brian Foy (Brault et al. 2004). This clone was modified by substituting the T7 promoter with an SP6 promoter and the removal of restriction sites. The PCR amplified fragments of CHIKV (37997) were produced using high fidelity PFU polymerase (Stratagene, La Jolla, Calif.). The fragments were ligated either singly or in tandem with T4 DNA ligase (Stratagene) and transformed into XL10-Gold cells (Stratagene). All plasmids were extracted using QIAprep Spin Miniprep Kit (Qiagen). The construction of the 5′ pCHIKic EGFP is illustrated in FIG. 1. The 3′ pCHIKic EGFP and the pCHIKic clones were constructed by similar methods. The 5′ and 3′ pCHIKic EGFP plasmids have the capacity to accept an insert of at least 724 bp in length using restriction sites AscI, PacI or EcoRI.

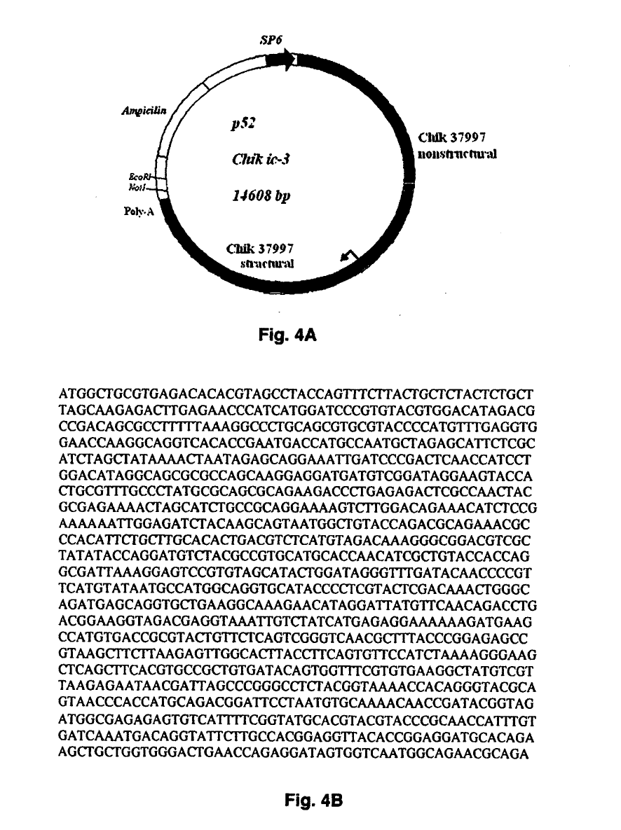

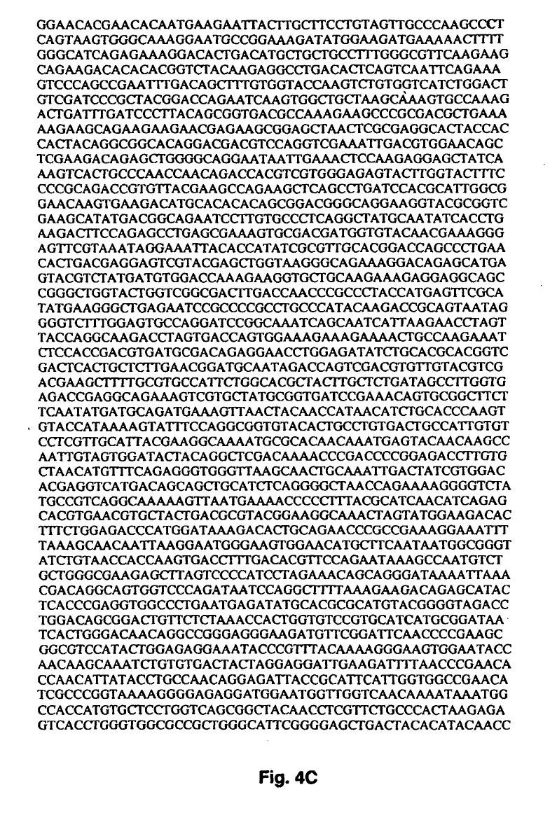

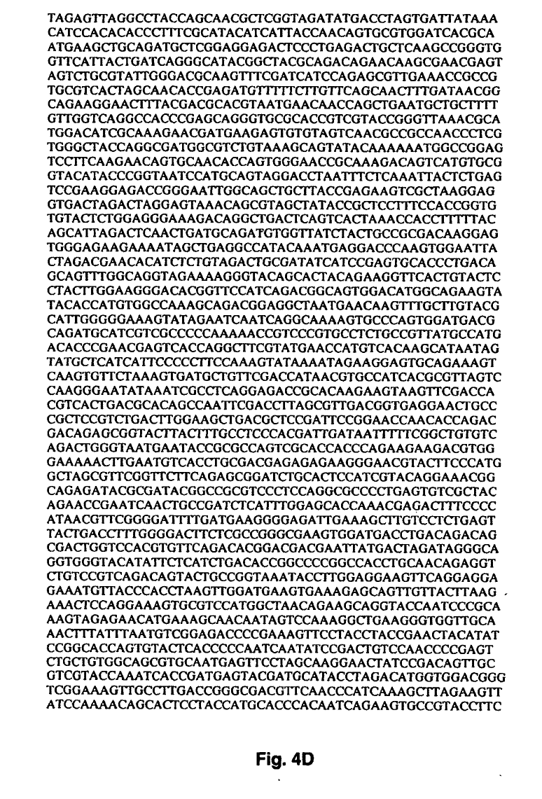

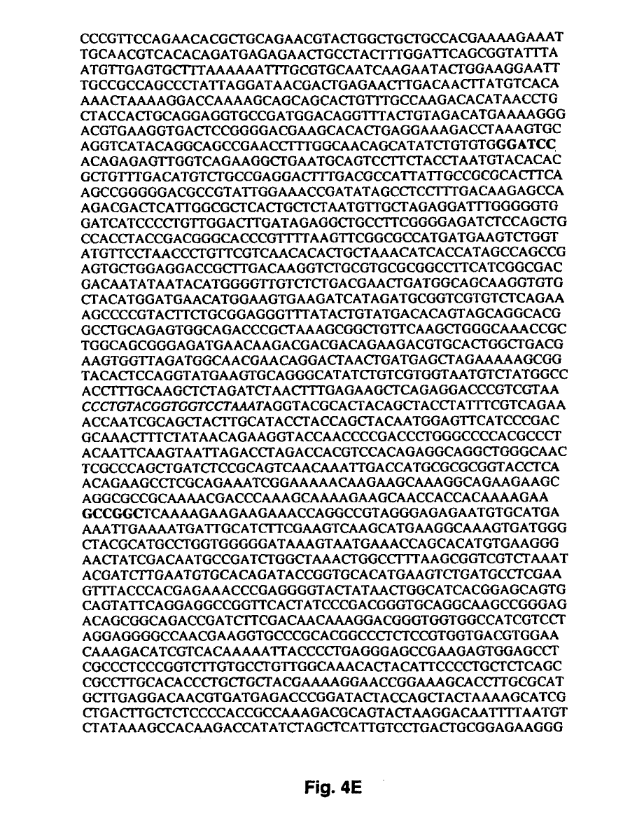

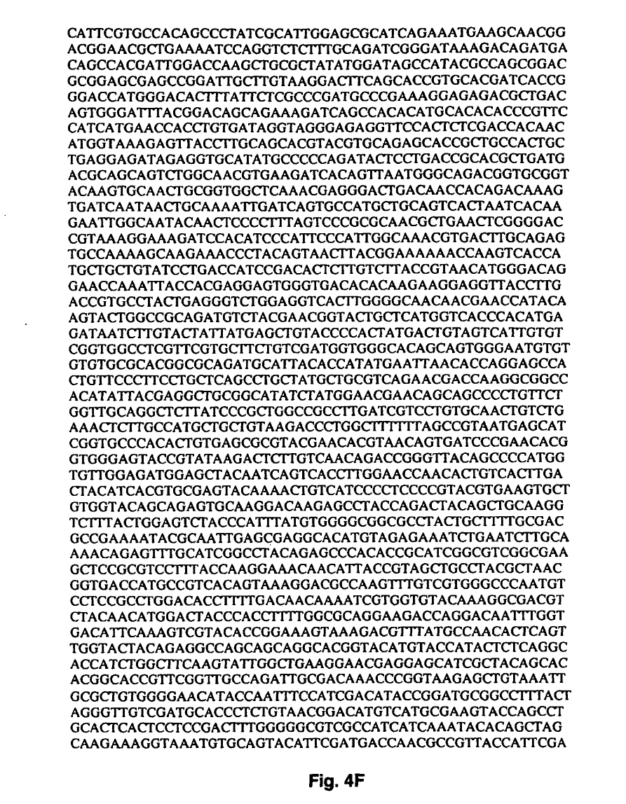

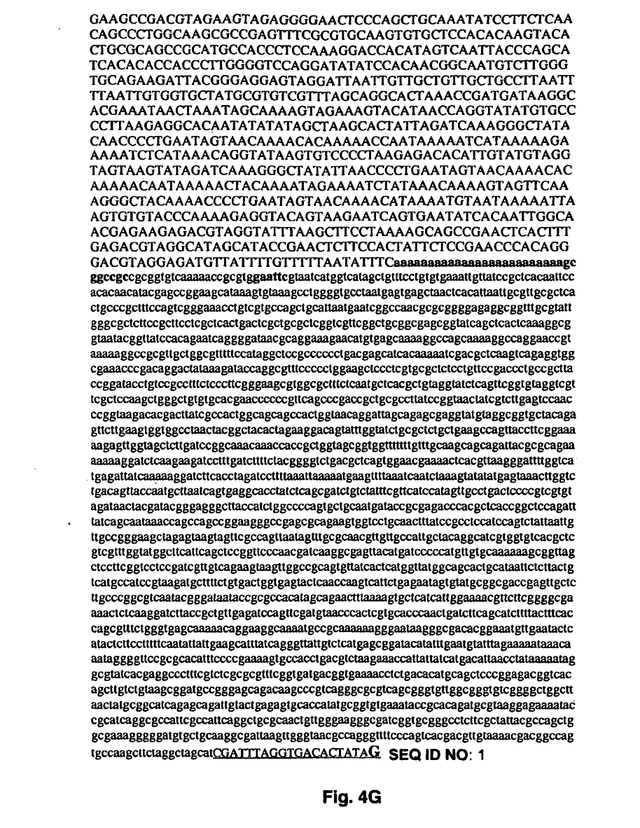

FIGS. 4A-4G show map and sequence of the plasmid: pChik-3 (SEQ ID NO: 1). Briefly, the plasmid was constructed as follows: The insert was amplified from p49.1 (pChik-2) using primers Chik-Sp6-F2 and Chik-Xma-R. The PCR product was digested with ClaI and XmaI restrictases and cloned into ClaI and XmaI sites of p49.1 (pChik-2). The resulting plasmid was named pChik-3 and one of the clones (clone 2) was partially sequenced from Chik-ns-R5. The sequence was 100% exact, the same as the Gene bank sequence of Chikungunya 37997 (AY726732), except two mutations in Ns-prot, which is indicated as X.

| | | | |
|---|---|---|---|
| Chik-Xma-R | 5′-TTAGGAATCCGTCTGCATG-3′ | 19 | 1002-1020* |
| | (SEQ ID No. 4) | | |
| Chik-Sp6-F2 | 5′-CTAGCATCGATTTAGGTGACACTATAG | 58 | 1-24* |
| | ATGGCTGCGTGAGACACACACGT-3′ | | |
| | (SEQ ID NO: 5) | | |

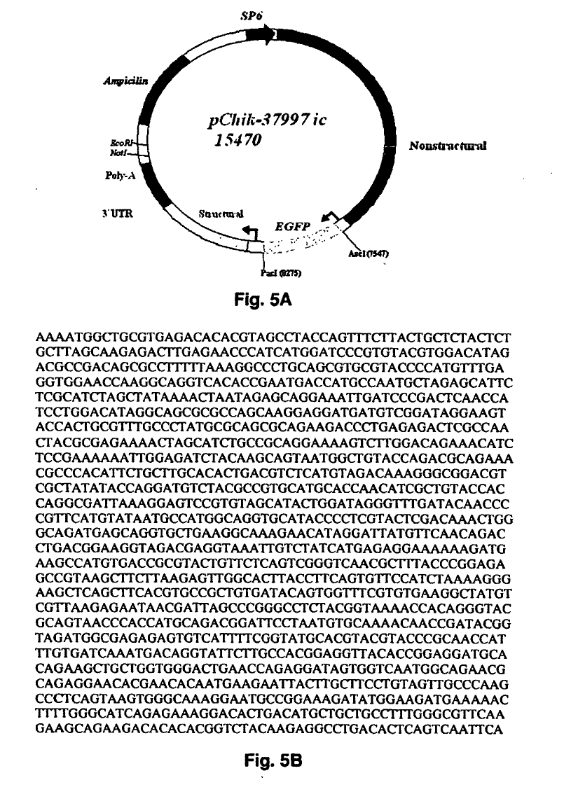

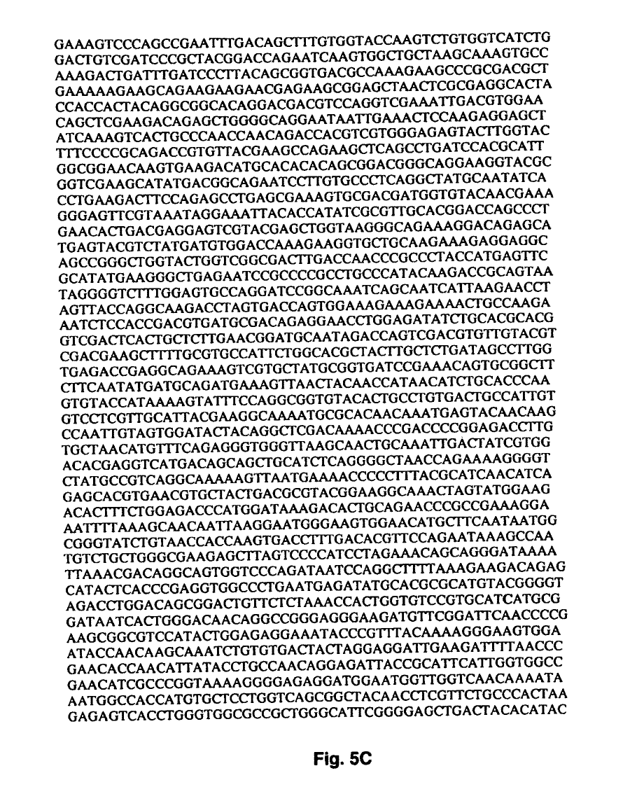

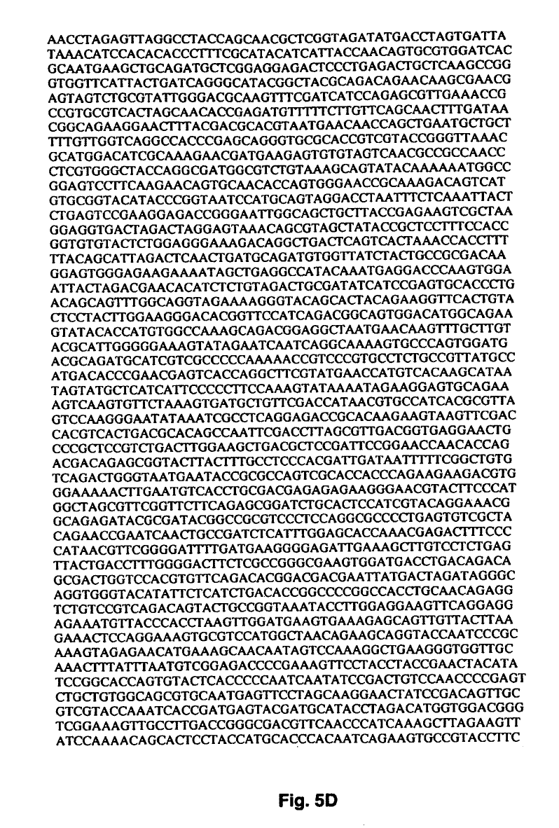

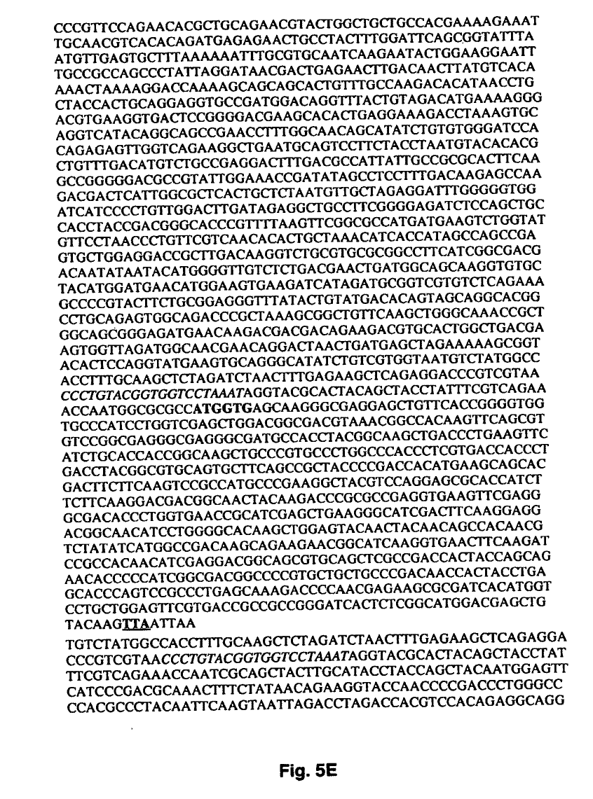

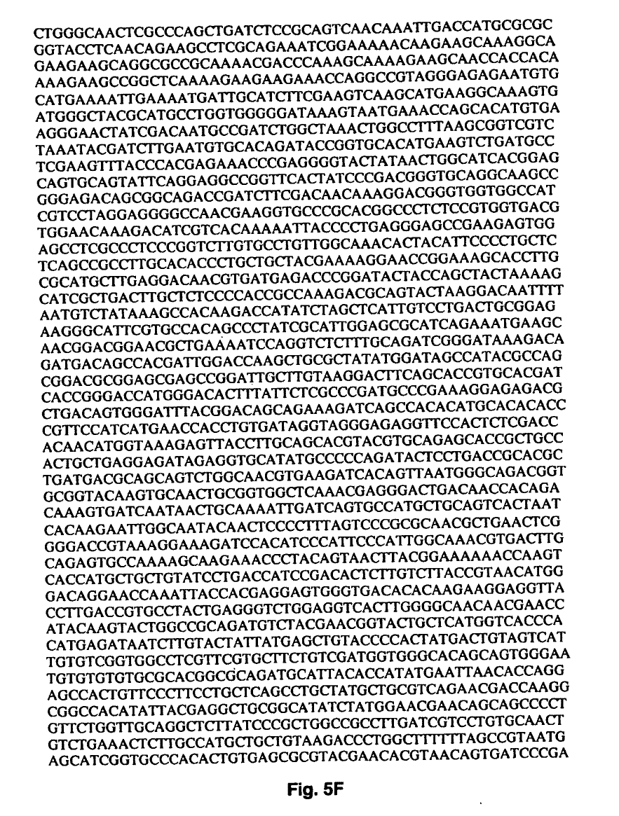

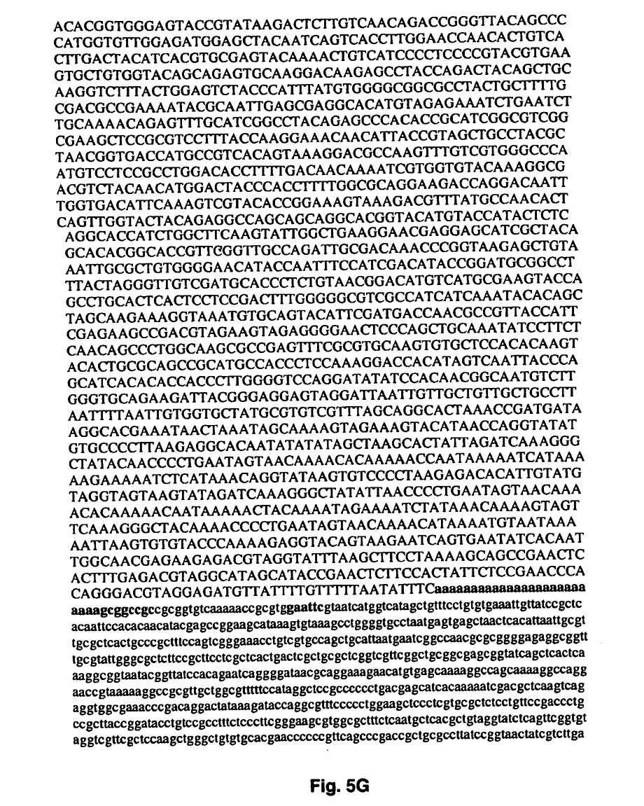

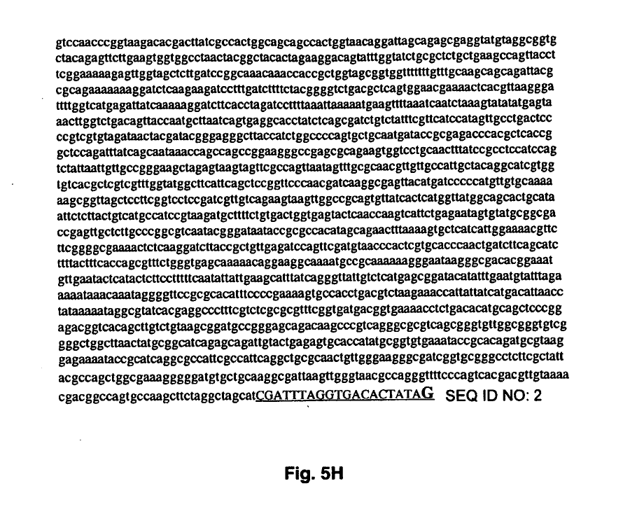

FIGS. 5A-5H show map and sequence of plasmid: p5′Chik-37997 is (SEQ ID NO: 2). Briefly the plasmid was constructed as follows: In order to clone 5′ region of Chik 37997, one fragment was amplified using primer set Chik-Sp6-F and Chik-Xma-R and digested with ClaI and XmaI restrictases. This fragment was cloned into ClaI and XmaI sites of the p27.1 (pOnnRep1-Chik-(XmaI-NotI). This clone was named pChikic and sequenced using Foy-F1 and Chik-Xma-R.

| | | | |
|---|---|---|---|
| Chik-Sp6-F | 5′-CTAGCATCGATTTAGGTGACACTATAG | 51 | 1-24* |
| | AAAATGGCTGCGTGAGACACACGT-3′ | | |
| | (SEQ ID NO: 5) | | |
| Chik-Xma-R | 5′-TTAGGAATCCGTCTGCATG-3′ | 19 | 1002-1020* |
| | (SEQ ID NO: 4) | | |
| Foy-F1 | 5′-TTTCCCAGTCACGACGTTGT3′ | 20 | 11587-11606 |
| | (SEQ ID NO: 6) | | in OnnREpl |

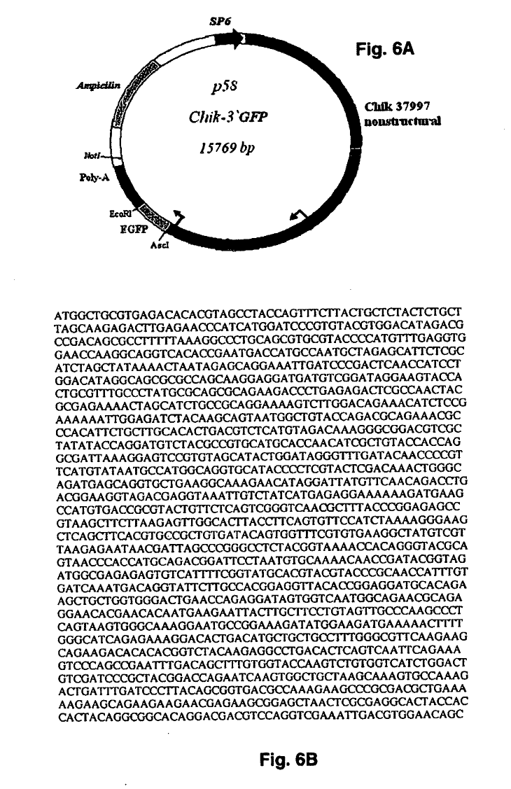

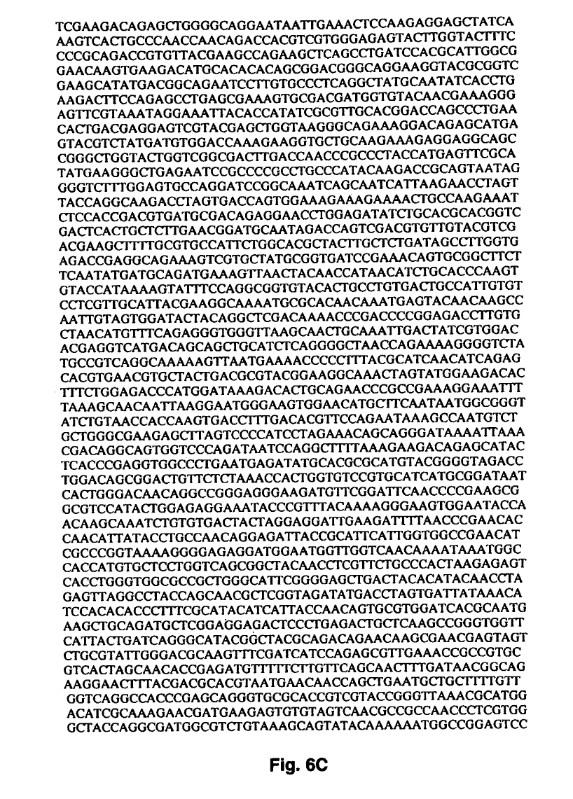

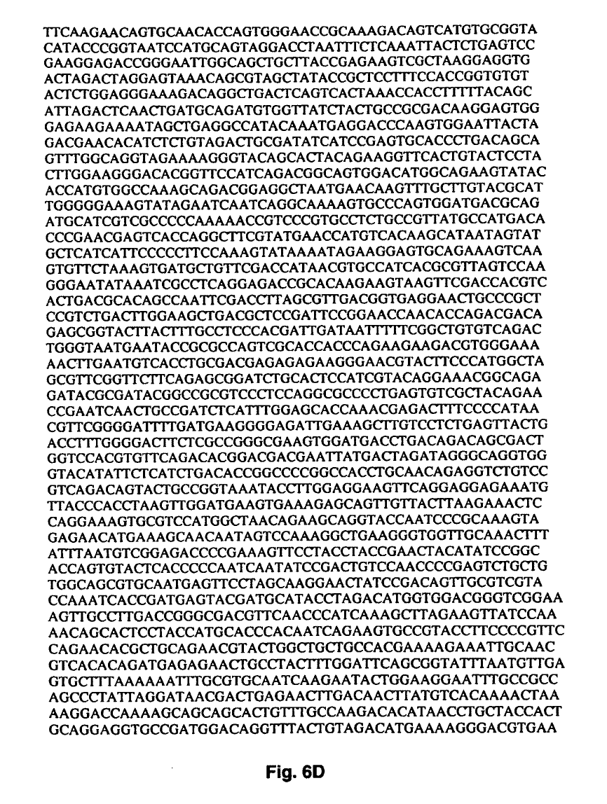

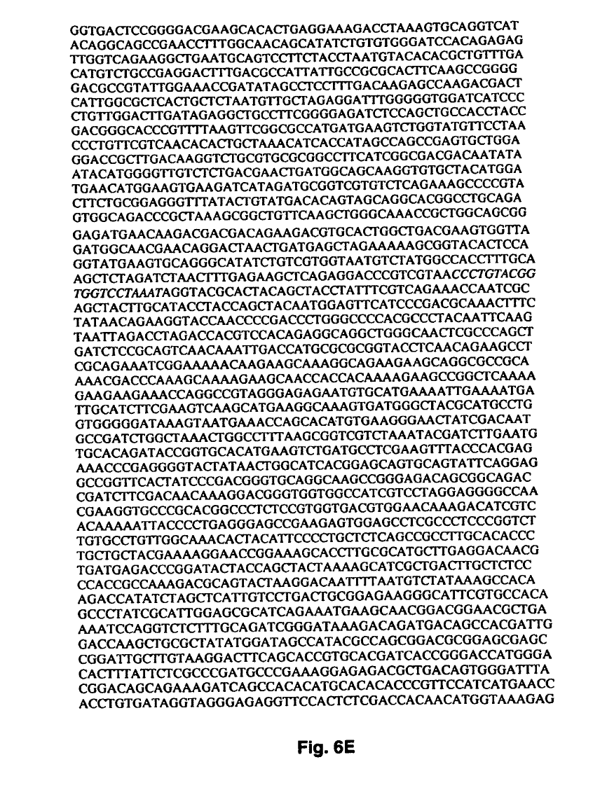

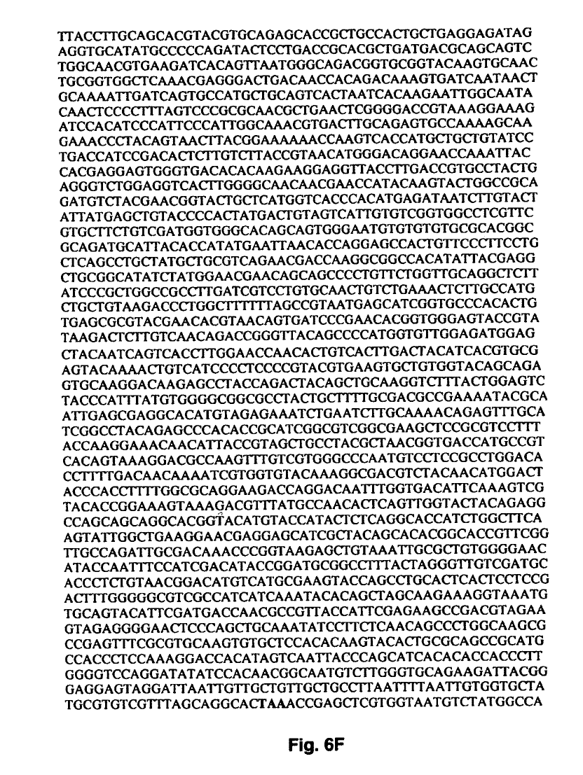

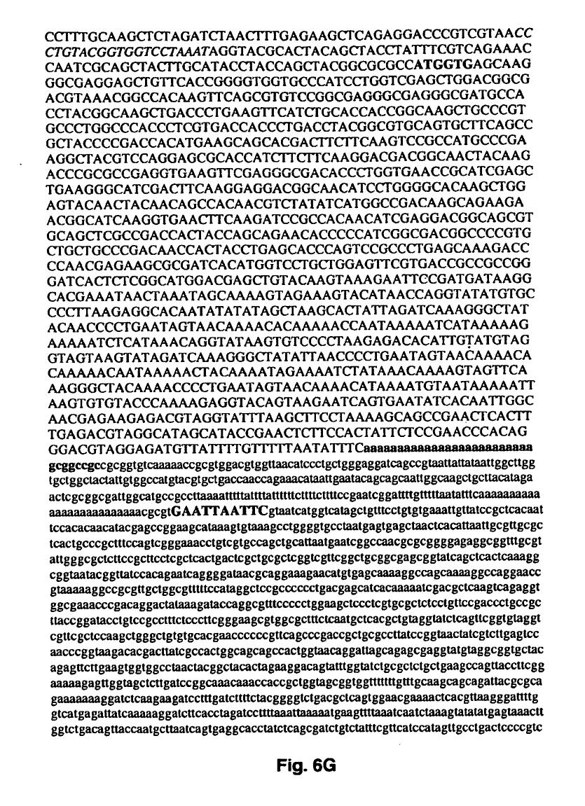

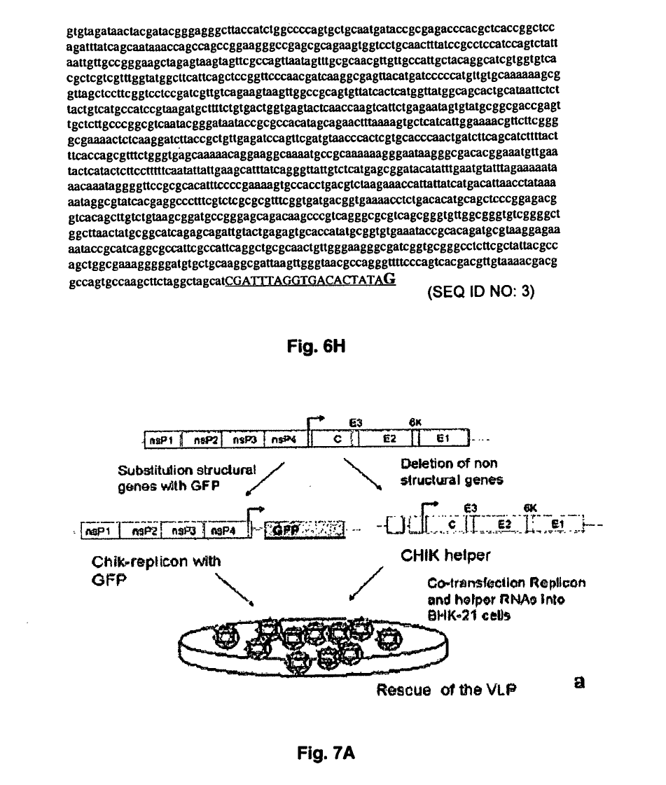

FIGS. 6A-6H show map and sequence of plasmid pChik-3′GFP (SEQ ID NO: 3). Briefly, this plasmid was constructed in two steps as follows: In the first step, an intermediate plasmid (pX) was constructed which was then used for cloning the complete construction. Step I: The plasmid X was made by simultaneous ligation and cloning of five DNA fragments. Fragment I was obtained by amplifying p52.2 (pChik-3) using primers Chik F3 and Chik-3UTR-Sac-R. The PCR product was digested with AseI and SacI. Fragment 2 was obtained by amplifying p52.2 (pChik-3) using primers Chik-Sac-F and Chik-EcoR-R. The PCR product was digested with SacI and EcoRI. Fragment 3 was obtained by amplifying p52.2 (pChik-3) using primers Chik-3UTR-EcoR-F and OnnRep1-R1. The PCR product was digested with EcoRI and NotI. Fragment 4 was obtained by cuting out a fragment (length=11270 base pairs) from p52.2 (pChik-3) with ClaI and AseI restrictases. Fragment 5(vector) was obtained by cuting out a fragment (length=2941 base pairs) from p38.1 (pOra+EcoR1) with NotI and Cla restrictases. The resulting plasmid was named pX.

Step 2: The insert was obtained by amplifying p26.1 (pChik-dSG-GFP) using primers Chik-Sac-F and GFP-EcoR-F. The PCR product was digested with EcoRI and AscI restrictases and cloned into sites EcoRI and AscI sites of the pX. This clone was then sequenced.

| | | | |
|---|---|---|---|
| Chik F3 | 5′-GGTGCAGAAGATTACGGGAG-3′ | 20 | 11225-11244* |
| | (SEQ ID NO: 7) | | |
| Chik-3UTR-Sac-R | 5′CAGTCGAGCTCGGTTTAGTGCCTGCTA-3 | 27 | 11303-11320 |
| | (SEQ ID NO: 8) | | |
| Chik-Sac-F | CAGTCGAGCTCGTGGTAATGTCTATGG | 27 | 7408-7427 |
| | (SEQ ID NO: 9) | | |
| Chik-EcoR-R | 5′-GCAGGAATTCGATATGGCGCGCC | 40 | 7551-7567 |
| | GTAGCTGGTAGGTATGC-3′ | | |
| | (SEQ ID NO: 10) | | |
| Chik-3UTR-EcoR-F | GCAGGAATTCCGATGATAAGGCACGAA | 27 | 11317-11334 |
| | (SEQ ID NO: 11) | | |
| OnnRepl-R1 | TCGGAATTCCACGCGGTTTTTGACAC | 26 | |
| | (SEQ ID NO: 12) | | |
| GFP-EcoR-F | GCAGGAATTCTTTACTTGTACAGCTCGT- | 28 | 992-1010 |
| | (SEQ ID NO: 13) | | |

### Example 6

### In Vitro Transcription of the pChikic Clones

Infectious virus from the pCHIKic clones (CHIKV, 5′ CHIKV EGFP, and 3′CHIKV EGFP) were produced by linearization with NotI which was in vitro transcribed from SP6 promoter using the mMESSAGE mMACHINE kit (Ambion) following manufacture's instructions. RNA was electroporated into BHK-21 S cells as previously described (Higgs et al. 1997). Cell culture supernatant containing virus was harvested, alliquoted and stored at −80° C. when the cells showed 75% CPE.

### Example 7

### In Vitro Growth Kinetics of Viruses

One vertebrate-derived, Vero and two mosquito-derived, C6/36 (Ae. albopictus) and MOS-55 (An. gambiae) cell lines were used for these studies. All cells were maintained in L-15 medium with 10% FBS, 100 U/mL penicillin, and 100 g/mL streptomycin. Vertebrate and mosquito cells were maintained at 37° C. and 28° C., respectively. CHIKV (37997) and infectious virus from pCHIKic (CHIKV) were grown on confluent cell monolayers, 25 cm² flasks were infected with a standard 1 mL inoculum by rocking at room temperature for 1 h. The inoculum was then removed and after three washes with 5 mL L-15, 5.5 mL of medium was added per flask. A sample of 0.5 mL was removed immediately. Additional 0.5 mL samples were collected at 24 h intervals and replaced with 0.5 mL of fresh medium. Samples were stored at −80° C. until titrated. Data represents virus production for a standardized monolayer area (25 cm²). Due to a difference in the size of individual cells, the multiplicity of infection varied for the different cell lines. Expression of EGFP was assessed following infection of 5′CHIKV EGFP and 3′ CHIKV EGFP in Vero and C6/36 cells using above discussed protocols. Viruses were compared at 48 h p.i. for the amount of EGFP expression.

### Example 8

### Mosquitoes

The white-eyed Higgs variant of the Rexville D strain of Ae. aegypti were reared at 27° C. and 80% relative humidity under a 16 h light: 8 h dark photoperiod, as previously described (Wendell et al. 2000; Miller and Mitchell 1991). Adults were supplied with a cotton wool pad soaked in a 10% sucrose solution ad libitum and fed on anaesthetized hamsters once per week for egg production.

### Example 9

### Virus Infections of Mosquitoes

Four day old adult female Ae. aegypti mosquitoes were fed a blood meal containing one of the four viruses to be analyzed. Fresh virus was grown from stock and harvested from Vero cells when 75% of the cells showed CPE. The viral supernatant was mixed with an equal volume of defibrinated sheep blood (Colorado Serum Company, Denver, Colo.). As a phagostimulant, adenosine triphosphate at a final concentration of 2 mM, was added to the blood meal.

Mosquitoes were fed using an isolation glove box located in a Biosafety Level 3 insectary. Infectious blood was heated to 37° C. and placed in a Hemotek feeding apparatus (Discovery Workshops, Accrington, Lancashire, United Kingdom) and mosquitoes were allowed to feed for 1 h (Cosgrove et al. 1994). Fully engorged females were separated from unfed females and were placed into new cartons. Three to eight mosquitoes were removed for titration on days 0, 1, 2, 3, 7, and 14 p.i. and were stored at −80° C. Day 0 samples, collected immediately after feeding, were used to determine the titer of virus imbibed and to evaluate continuity between experiments.

### Example 10

### Titrations

Viral samples harvested from cell culture and mosquitoes were quantified as tissue culture infectious dose 50 endpoint titers (log₁₀ TCID₅₀/mL) using a standardized procedure (Higgs et al. 1997). Briefly, 100 L samples of cell culture supernatant/mosquito triturate were pipetted into wells of the first column of a 96-well plate, serially diluted in a 10-fold series, seeded with Vero cells and incubated at 37° C. for seven days. Prior to titration, each mosquito was tritrated in 1 mL of L-15 medium and filtered through a 0.22M syringe filter (Millipore, Carrigwohill, Cork, Ireland).

### Example 11

### Immunofluorescence Assay (IFA) and EGFP Analysis

Midguts and salivary glands were dissected from 7 and 14 day p.i. mosquitoes for analysis to determine dissemination rates. The mosquitoes were dissected on glass microscope slides in phosphate buffered saline. For IFA, salivary glands were air dried, fixed in cold acetone for 10 min and stained using a cross-reactive mouse hyperimmune ascitic fluid raised against chikunguna virus as the primary antibody and amplifying the signal using indirect IFA protocols described (Gould et al. 1985a; Gould et al. 1985b; Higgs et al. 1997).

For analysis of EGFP expression, midguts and salivary glands were dissected directly into glycerol-saline and immediately examined for EGFP expression under an Olympus IX-70 epifluorescence microscope. Differences in the infection and dissemination rates based on IFA or EGFP analysis were tested for significance using Fisher's Exact Test, SPSS version 11.5 (SPSS Inc. Chicago, Ill.).

### Example 12

### Results

Chikungunya virus (37997) and chikungunya virus derived from pCHIKic in the vertebrate cell line, Vero and two invertebrate cell lines, C6/36 (Ae. albopictus) and MOS-55 (An. gambiae), displayed similar in vitro growth characteristics (FIG. 2). The peak titer of both chikungunya virus (37997) and chikungunya virus in Vero and C6/36 cells was reached at day 2 p.i. The titers decreased at similar rates from day 2 p.i. to day 6 p.i. (FIG. 2). 5′CHIKV EGFP and 3′CHIKV EGFP were compared in Vero and C6/36 cells to assess the levels of EGFP expression in cell culture. The 3′ clone expressed EGFP at a markedly higher intensity than the 5′ clone in both cell types examined.

In vivo experiments were conducted in Ae. aegypti mosquitoes to compare the CHIKV (37997) and the three viruses derived from infectious clones. The blood meal titers for the CHIKV (37997) and chikungunya virus were identical, 7.95 log₁₀TCID₅₀/mL and the percent of infected mosquitoes and titers of virus in the mosquitoes were similar by whole body titrations of mosquitoes at six time points p.i. (Table 2). The two clones that expressed EGFP had slightly lower blood meal titers when compared to the chikungunya virus. Both the 5′ and the 3′ CHIKV EGFP had a blood meal titer of 7.52 log₁₀TCID₅₀/mL. Although the blood meal titers were slightly different between the viruses with or without EGFP, all of the viruses infected 100% of the mosquitoes on day 14 p.i. (Table 2).

IFA and EGFP were used to determine the percent of mosquitoes infected on days 7 and 14 p.i. (Table 3). IFA and EGFP data were compared using dissected midguts to determine infection rates and dissected salivary glands to determine dissemination rates. Expression of EGFP in the eyes of day 14 p.i. Ae. aegypti indicated infected nervous tissue, virus was not observed in other tissues. Two experiments were completed to compare virus derived from the 5′ and 3′ pCHIKic EGFP constructs (Table 3). In both experiments, the infection rates were 100% on days 7 and 14 p.i. for both viruses (Table 3). The dissemination rates were similar based on antigen detection by IFA and by EGFP visualization for the 5′ CHIKV EGFP with 100% and 90% dissemination by IFA and EGFP, respectively (Table 3). The 3′ CHIKV EGFP dissemination rates on day 14 p.i. were different for the two experiments. The percent of mosquitoes with disseminated infections by IFA were 100% on day 14 p.i. where as the percent of mosquitoes with disseminated infections by EGFP were 70% (Table 3).

### Example 13

### Development of a CHIKV (37997) Replicon System

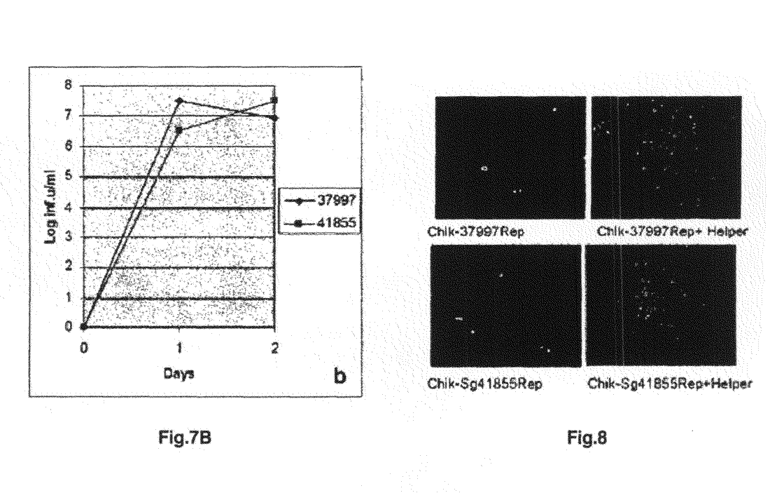

The full-length infectious clones for CHIKV (37997) and SG1855 have been characterized in vitro and in vivo in Ae. aegypti and in various cell types as discussed supra. These clones were used as a backbone to construct replicon and helper system for Ae. aegypti and Ae. albopictus mosquitoes (FIG. 7A). To simplify detection of replication events in the replicon infected cells, EGFP was introduced into both replicons under the control of a viral subgenomic promoter. The dynamics of the accumulation of CHIKV infectious units in BHK-21 cells that were co-transfected with CHIKV replicon and helper RNA is shown in FIG. 7B.

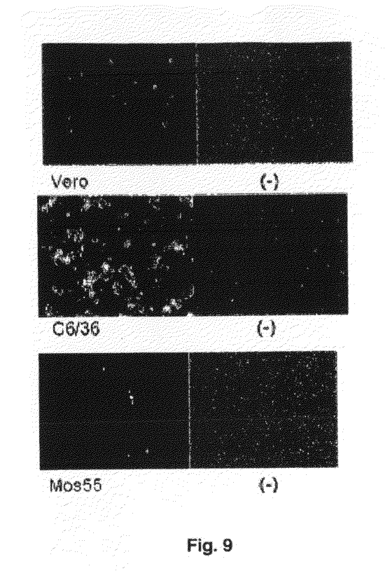

Transfection of BHK-21 cells with CHIKV replicon RNA alone provided expression of viral replicase which produced intense GFP fluorescence at 8 h post-transfection (FIG. 8). No virus like particles (VLP) were generated because the replicon was unable to package itself. Therefore, GFP was expressed only in primary infected cells without spreading to neighboring cells. Foci of EGFP-expressing cells, following co-transfection of BHK-21 cells with both replicon and helper RNA, indicate active packaging of replicon RNA into VLPs that are capable of infecting adjacent cells. Additionally, Vero, C6/36 and Mos55 cell types were infected with CHIK 37997 VLPs with multiplicity of infection (MOI) 1, as determined on Vero cells (FIG. 9). CHIKV 37997 VLPs efficiently infected Vero and C6/36 cells and were less infectious in Mos 55 cells. This observation correlated with the infection patterns for the original virus, CHIKV 37997 (Vanlandingham et al., 2005). These data indicate that CHIK VLP produced from the replicon system possessed similar cellular tropisms as the original virus and could be used as a convenient tool for either identification of sites of primary CHIKV infection in mosquito vectors or for identification of cellular receptor molecules for CHIKV in mosquito vectors.

### Example 14

### Vector Competence Studies for Infectious Clones of Chikungunya Virus (La Reunion Isolate)

Virus: The LR2006 OPY1 strain of CHIKV (CHIKVLR) was obtained from the World Reference Center for Arboviruses at the University of Texas Medical Branch, Galveston, Tex. This strain was originally isolated from serum of a febrile French patient returning from La Réunion Island. The strain was passed five times on Vero cell culture and once in suckling mice. Stock virus was produced following a single passage in C6/36 cells, grown at 28° C. in Leibovitz L-15 media with 10% FBS and stored at −80° C. until needed.

Infectious clone production: The CHIKV full-length infectious clone (CHIKV-LR ic) was produced using standard molecular biology techniques (FIG. 10A) (Vanlandingham et al. 2005a). The QIAamp Viral RNA Mini kit (Qiagen, Valencia, Calif.) was used for RNA extraction from viral stock which was then reverse transcribed from random hexanucleotide or oligo-dT primers (Promega, Madison, Wis.) using Superscript II reverse transcriptase (Invitrogen Life Technologies). CDNA was amplified using Pfu-turbo DNA polymerase (Stratagene, La Jolla, Calif.) and PCR fragments were cloned in tandem into a full length infectious clone using a modified pSinRep5 plasmid backbone (Invitrogen Life Technologies). The SP6 promoter sequence was introduced upstream of the 5′ end of CHIKV cDNA sequence, and the viral poly A₄₀ tail and NotI linearization site was added to the 3′ end. To ensure that no mutations were introduced during the cloning procedures at least two clones of the intermediate and final plasmids were sequenced completely.

Two double genomic CHIKV infectious clones were also developed which express green fluorescent protein (GFP). In the first construct, CHIKV-LR 5′GFP, GFP is expressed 5′ to the structural genes of the viral cDNA from a second subgenomic promoter. In the second construct, CHIKV-LR 3′GFP, GFP is expressed from a subgenomic promoter located at the 3′ end of the structural genes of viral cDNA (FIG. 10B). The sequences of the primers and infectious clones are not provided here but please provide the sequences.

RNA transfections and infectious center assays: Infectious virus from the pCHIKic clones (CHIKV-LR ic, CHIKV-LR 5 GFP, and CHIKVLR-3 GFP) were produced by linearization with NotI. The NotI was in vitro transcribed from SP6 promoter using the mMESSAGE mMACHINE kit (Ambion). 10 μg of RNA was electroporated into 1×10⁷ BHK-21 cells as described (Higgs et al. 1997; Vanlandingham et al. 2005b). The cells were than seeded into 75-cm² flask in 15 mL of L-15 media. At 24 and 48 h post-electroporation, 10 mL of tissue culture medium was harvested, titrated, and stored in aliquots at 80° C. Serial tenfold dilutions of electroporated BHK-21 were seeded in six-well tissue culture plates containing 5 10⁵ naive BHK-21 per well for infectious center assays. Following incubation for 2 h at 37° C., cells were overlaid with 2 mL of 0.5% agarose containing L-15 supplemented with 3.3% FBS. BHK-21 cells were incubated for 2 days at 37° C. until plaques developed and were stained with crystal violet.

In vitro growth of virus: Vertebrate and mosquito cells were maintained at 37° C. and 28° C., respectively, in Leibovitz L-15 medium with 10% FBS, 100 U/mL penicillin, and 100 g/mL streptomycin. Confluent monolayers of Vero (green monkey) and C6/36 (Ae. albopictus) cells were infected with each virus at a multiplicity of infection (moi) of 0.1 by rocking for 1 h at 25° C. in 25-cm2 flasks. Cells were washed with 5 mL of L-15 medium three times and 5.5 mL of L-15 was added per flask. At day 0 and at 12, 24, 48, 72, and 96 h post-infection (p.i.), a 0.5-mL sample of medium was removed and stored at 80° C. The volume of medium was then restored by adding 0.5 mL of fresh medium. All data represent virus production for a standardized monolayer area (25 cm²).

Titrations: Viral samples harvested from cell culture and mosquitoes were quantified as tissue culture infectious dose 50 endpoint titers (log₁₀ TCID₅₀/mL) using a standard procedure (Higgs et al., 1997). 100 μL samples of cell culture supernatant medium/mosquito triturate were pipetted into wells of the first column of a 96-well plate, serially diluted in a 10-fold series, seeded with Vero cells and incubated at 37° C. for 7 days. Prior to titration, each mosquito was triturated in 1 mL of L-15 medium and centrifuged for 5 min at 10,000 rpm.

Stability assay: Stability of GFP expression of double subgenomic viruses was evaluated as previously described (Brault et al. 2004). Monolayers of BHK-21 and C6/36 cells in 25-cm2 flasks were initially infected with CHIKV-LR 5 GFP, or CHIKV-LR-3 GFP at 0.1 moi and maintained in 6 mL of L-15. At 2 days after infection, 2 μL of tissue culture supernatant medium from BHK-21 cells and 10 μL of supernatant from C6/36 were used to infect 25-cm² flasks of fresh BHK-21 and C6/36 cell cultures. These serial passages were performed ten times for BHK-21 cells and eight times for C6/36 cells. At the end of each passage, the percentage of cells in the flask expressing GFP was estimated by viewing the cells through an Olympus IX51 epifluorescence microscope. Additionally, cell culture supernatant medium from each virus passage was titrated for virus by standard plaque assay. The plates were first analyzed by fluorescence microscopy to determine the titer of GFP positive-foci followed by staining with neutral red to determine the number of plaque forming units.

Virus infection of mosquitoes: Each of the four viruses to be characterized was fed to four to five day old female Ae. aegypti (Rexville D-Higgs white-eye strain) and Ae. albopictus (Galveston strain) mosquitoes using an artificial infectious blood meal. The blood meal was produced using stock virus grown on C6/36 cells harvested at 2d p.i. mixed with equal volumes of defibrinated sheep blood (Colorado Serum Company, Denver, Colo.). Mosquitoes were infected as previously described (Vanlandingham et al. 2005a,b, 2006) using an isolation glove box located in a Biosafety Level 3 insectary. Infectious blood was heated to 37° C. and placed in a Hemotek feeding apparatus (Discovery Workshops, Accrington, Lancashire, UK) (Cosgrove et al. 1994), and mosquitoes were allowed to feed for 45 min. Unfed females were discarded and fully engorged females were transferred to new cartons. Three to 16 mosquitoes were removed for titration on days 0, 1, 3, 7, and 14 p.i. and were stored at 80° C. Day 0 samples were collected immediately following feeding and were used to determine the titer of virus imbibed. Five to 10 Ae. aegypti or Ae. albopictus, infected with either CHIKV-LR 5′ GFP or CHIKV-LR-3′ GFP, were cold anesthetized and midguts and salivary glands were dissected for analysis of GFP expression on days 7 and 14 p.i. as previously described (Vanlandingham et al. 2005a). Dissemination rates were calculated as a ratio between GFP positive salivary glands over positive midguts.

Results: The plasmid pSinRep5 (Invitrogen) was used as a backbone for the construction of a full length infectious clone of the La Réunion island isolate, CHIKV LR2006 OPY1 strain (CHIKVLR). Five overlapping cDNA fragments were cloned together using natural restriction sites. This clone has an SP6 promoter at the beginning of the viral genome and a Nod linearization site immediately following the poly A40 tail at the 3 end of the viral cDNA. The construct was designated CHIKV-LR ic (FIG. 10A) and two independent clones were completely sequenced. Sequences of both clones were identical to each other but differed at six positions (1053 G→A, 4168 A→G, 5050 T→G, 6608 A→G, 6614 C→A, 6623 A→G) as compared with the LR2006 OPY1 coding sequence available in GenBank (DQ443544). Additionally this sequence (DQ443544) had an insertion of a T in the third position of the viral genome and a deletion of 44 nucleotides beginning at position 11,696 in the 3 untranslated region and another deletion of the last five nucleotides adjacent to the poly A tail.

The quality of the infectious clones produced was analyzed by two methods: specific infectivityinfectivity of the RNA, and viral titers of rescued virus, following electroporation of the RNA into BHK-21 cells (Table 4). These experiments demonstrated that the in vitro transcribed RNA is capable of initiating the viral replication cycle which ultimately leads to the production of mature virus. Comparison of the growth kinetics of CHIKV-LR and virus produced from CHIKV-LR ic revealed similar growth curves on vertebrate and mosquito cell culture (FIGS. 11A-11B). Maximum titers of 7.52 log₁₀TCID₅₀/mL in Vero cells and 8.52 log₁₀TCID₅₀/mL in C6/36 cells were reached after 24 and 48 h, respectively (FIGS. 11A-11B).

Infection rates of the CHIKV-LR and the virus derived from the full length CHIKV-LR ic were compared in Ae. aegypti and Ae. albopictus mosquitoes and analyzed by 10-fold serial dilution in 96 well plates. The average blood meal titer of CHIKV-LR and virus derived from CHIKV-LR ic in Ae. aegypti were the same, 7.24±0.4 log₁₀TCID₅₀/mL (Table 5). For Ae. albopictus, the blood meal titers of CHIKV-LR and virus derived from CHIK-LR ic were 7.24±0.4 log₁₀TCID₅₀/mL and 6.95±0.0 log₁₀TCID₅₀/mL, respectively. Viruses were found to be infectious to both species of mosquitoes, CHIKV-LR infecting 78% whilst virus derived from CHIKV-LR ic infected 60% of Ae. aegypti mosquitoes. Both viruses infected 94% of Ae. albopictus examined on day 14 p.i. (Table 5).

TABLE 4
Specific infectivity of RNAs in vitro transcribed from CHIKV-LR IC,
CHKV-LR 5′ GFP and CHIKV-LR 3′GFP infectious clones.

| | Virus titer^{a} (log₁₀TCID₅₀/mL) | | |
|---|---|---|---|
| Template used for in | Specific infectivity | | |
| vitro transcription | (pfu/μg of RNA) | 24 h | 48 h |
| CHIK-LR ic | 8.0 × 10⁵ | 6.95 | 7.95 |
| CHIK-LR 5′GFP | 4.8 × 10⁵ | 6.52 | 6.52 |
| CHIK-LR 3′GFP | 3.2 × 10⁵ | 8.52 | 7.95 |

^{a}Virus titer from cell culture supernatant medium at 24 and 48 h post-electroporation.

Specific infectivity and viral titer in vitro were used to determine the quality of the double subgenomic promoter infectious clones which express GFP (Table 4). Specific infectivity of RNA produced from both the CHIKV-LR5′ GFP and CHIKV-LR 3′GFP infectious clones were similar to each other and were slightly lower than the specific infectivity of the RNA produced from CHIKV-LR ic. This indicates that no lethal mutations were introduced into the viral genome during construction of these plasmids (Table 4). GFP expression in BHK-21 cell culture transfected with RNA derived from both double sub-genomic GFP-expressing constructs was detected at 3-4 h post-electroporation and reached its maximum density at 15-24 h post-electroporation. The level of GFP expression was considerably higher for the CHIKV-LR 3′ GFP construct (FIG. 10C). Growth kinetics of both viruses expressing GFP were analyzed in Vero and C6/36 cell cultures (FIGS. 11A-11B). The viruses produced similar growth curves in both cell lines, reaching peak titers at day 2 p.i. for Vero and day 1 p.i. for C6/36 cells. Interestingly, both GFP expressing viruses grew less efficiently on Vero cells as compared with CHIKV-LR and virus produced from CHIKVLR ic (FIG. 11A). The growth kinetics of the CHIKV-LR 5 GFP and CHIKV-LR 3 GFP on C6/36 cells indicated a more prominent attenuated pattern. The peak titer of both GFP viruses was at least one log₁₀ TCID₅₀/mL lower in C6/36 cells as compared with CHIKV-LR and virus derived from CHIKV-LR ic (FIG. 11B).

TABLE 5
Infection rates and average titer of ae. aegypti and ae. albopictus mosquitoes
orally infected with CHIKV-LR or CHIKV-LR ic.

| | | CHIKV-LR^{a} | | CHIKV-LR ic^{b} | |
|---|---|---|---|---|---|
| | | Infected/total | | Infected/total | |
| Mosquito | Day p.i. | (%) | Titer^{c} ± SD | (%) | Titer^{c} ± SD |
| Ae. aegypti | 0 | 6/6 (100) | 5.31 ± 0.42 | 6/6 (100) | 5.74 ± 0.67 |
| | 1 | 6/6 (100) | 4.40 ± 2.45 | 6/6 (100) | 4.38 ± 0.57 |
| | 3 | 5/5 (100) | 5.32 ± 0.45 | 7/8 (88) | 4.91 ± 0.77 |
| | 7 | 11/15 (73) | 5.50 ± 0.73 | 14/18 (78) | 6.13 ± 0.78 |
| | 14 | 14/18 (78) | 5.27 ± 0.64 | 6/10 (60) | 5.64 ± 0.36 |
| Ae. albopictus | 0 | 6/6 (100) | 4.21 ± 0.60 | 6/6 (100) | 4.62 ± 0.27 |
| | 1 | 5/5 (100) | 5.18 ± \grave{ }.26 | 4/4 (100) | 6.02 ± 0.58 |
| | 3 | 4/4 (100) | 4.96 ± 0.52 | 6/6 (100) | 3.90 ± 1.32 |
| | 7 | 17/18 (94) | 4.90 ± 0.69 | 15/16 (94) | 5.08 ± 0.78 |
| | 14 | 17/18 (94) | 3.75 ± 0.69 | 15/16 (94) | 4.35 ± 0.58 |

^{a}Titer of CHIKV-LR blood meals fed to Ae. aegypti and Ae. albopictus: 7.24 ± 0.4 log₁₀ TCID₅₀/mL.
^{b}Titer of CHIKV-LR ic blood meals fed to Ae. aegypti and Ae. albopictus: 7.24 ± 0.4 log₁₀ TCID₅₀/mL and 6.95 ± 0.0 log₁₀ TCID₅₀/mL, respectively.
^{c}Titer reported as log₁₀ TCID₅₀/mL.

CHIKV-LR 5′GFP and CHIKV-LR 3′GFP viruses were serially passaged in a vertebrate (BHK-21) and an invertebrate (C6/36) cell line in order to assess the stability of the expression of the gene of interest (GOI), GFP, under the control of the second sub-genomic promoter (Table 6, FIGS. 12A-12B). Previously, alphavirus expression vectors can be unstable and lose their ability to express the GOI during serial passage in cell culture (Pugachev et al. 1995, Brault et al. 2004). For each passage, the percentage of cells expressing GFP was determined (Table 6). In BHK-21 cell cultures, recombinant viruses were relatively stable and retained the ability to drive GFP expression in at least 85% of the cells after six serial passages. The CHIKV-LR 5′GFP was more stable and demonstrated slower kinetics of GFP loss as compared with CHIKV LR 3′ GFP. After 10 passages in BHK-21, the CHIK-LR 5′ GFP and CHIK-LR 3′ GFP viruses retained the ability to express GFP in approximately 50% and 5% of the cells, respectively (Table 6). The same pattern was observed during serial passage of both viruses on C6/36 cells; after six passages only 10% of C6/36 cells infected with virus derived from CHIKV-LR 3 GFP were able to express GFP whilst 85% of the cells infected with virus derived from CHIKV-LR 5 GFP were positive for GFP (Table 6). Similar results were obtained in experiments in which the titer of plaque forming units (pfu) was compared with the titer of GFP foci forming units (F) of viruses collected after each passage (FIGS. 12A-12B). For CHIKV-LR 3′ GFP in BHK-21, the difference between fluorescent titer and titer based on cytopathic effect (CPE) was less then 0.5 log₁₀ during the first eight passages and reached a difference of 2 log₁₀ at passage 10. In C6/36 cell culture a 2 log₁₀ difference was reached at passage six.

TABLE 6
Stability of GFP expression induced by CHIKV-LR 5′ GFP and
CHIKV-LR 3′ GFP viruses after serial passage in BHK-21 and
C6/36 cell culture.

| | Percentage of cells expressing GFP | | | | | | | | | |
|---|---|---|---|---|---|---|---|---|---|---|
| Virus | P1 | P2 | P3 | P4 | P5 | P6 | P7 | P8 | P9 | P10 |
| BHK cells | | | | | | | | | | |
| CHIK- | 100 | 100 | 99 | 98 | 95 | 90 | 90 | 85 | 80 | 50 |
| LR 5′GFP | | | | | | | | | | |
| CHIK- | 100 | 99 | 99 | 95 | 80 | 85 | 65 | 50 | 30 | 5 |
| LR 3′GFP | | | | | | | | | | |
| C6/36 cells | | | | | | | | | | |
| CHIK- | 100 | 95 | 95 | 90 | 87 | 85 | 83 | 80 | ND | ND |
| LR 5′GFP | | | | | | | | | | |
| CHIK- | 100 | 90 | 90 | 70 | 50 | 10 | 2 | <1 | ND | ND |
| LR 3′GFP | | | | | | | | | | |

Viruses derived from the 5′ and 3′ CHIKVLR GFP ic were compared in Ae. aegypti and Ae. albopictus mosquitoes (Table 7). Mosquitoes were fed virus derived from CHIKV-LR 5 GFP ic or CHIKV-LR 3 GFP ic. The titers of the blood meals were 5.52 log₁₀TCID₅₀/mL for CHIKV-LR 5′GFP and 6.27 log₁₀TCID₅₀/mL for CHIKV-LR 3 GFP. Viruses derived from CHIKV-LR 5′GFP and CHIKV-LR 3′GFP infected 63% and none (0/8) of the Ae. aegypti, respectively, when examined on day 14 p.i. (Table 7). Infection rates of Ae. albopictus infected with virus derived from CHIKV-LR 5′GFP and CHIKV-LR 3′GFP were 100% and 63% on day 14 p.i. respectively (Table 7). Ae. aegypti and Ae. albopictus mosquitoes were examined by GFP analysis in the midguts and salivary glands. Virus derived from the CHIKV-LR 5 GFP ic infected 100% of midguts and disseminated in 88% of the Ae. aegypti and Ae. albopictus examined on day 14 p.i. (Table 7). Virus derived from the CHIKV-LR 3 GFP is did not infect Ae. aegypti mosquitoes at day 7 or 14 p.i. However, this virus did infect 90% of the Ae. albopictus mosquitoes but only one salivary gland was observed to be positive for GFP on day 14 p.i. (Table 8).

TABLE 7
Infection rates and average titer of Ae. aegypti and Ae. albopictus mosquitoes
orally infected with CHIKV-LR 5′GFP or CHIKV-LR 3′GFP.

| | | CHIKV-LR 5′GFP^{a} | | CHIKV-LR 3′GFP^{b} | |
|---|---|---|---|---|---|
| | | Infected/total | | Infected/total | |
| Mosquito | Day p.i. | (%) | Titer^{c} ± SD | (%) | Titer^{c} ± SD |
| Ae. aegypti | 7 | 8/8 (100) | 5.25 ± 0.39 | 0/8 | 0 |
| | 14 | 5/8 (63) | 4.78 ± 0.74 | 0/8 | 0 |
| Ae. albopictus | 7 | 8/8 (100) | 4.36 ± 0.51 | 7/8 (88) | 3.99 ± 1.49 |
| | 14 | 7/7 (100) | 3.81 ± 0.53 | 4/4 (100) | 4.69 ± 0.24 |

^{a}Titer of CHIKV-LR 5′ GFP blood meals fed to Ae. aegypti and Ae. albopictus: 5.52 log₁₀ TCID₅₀/mL.
^{b}Titer of CHIKV-LR 3′ GFP blood meals fed to Ae. aegypti and Ae. albopictus: 6.27 log₁₀ TCID₅₀/mL.
^{c}Titer reported as log₁₀ TCID₅₀/mL.

TABLE 8
Expression of GFP in Ae. aegypti and Ae. albopictus mosquitoes orally infected
with CHIKV-LR 5′GFP or CHIKV-LR 3′GFP.

| | | CHIKV-LR^{a} | | CHIKV-LR ic^{b} | |
|---|---|---|---|---|---|
| | | Positive | Positive SG | Positive | Positive SG |
| | | midguts/total | positive mid | midguts/ | positive mid |
| Mosquito | Day p.i. | (%) | guts (%) | total (%) | guts (%) |
| Ae. aegypti | 7 | 5/8 (63) | 5/5 (100) | 0/8 | 0 |
| | 14 | 8/8 (100) | 7/8 (88) | 0/8 | 0 |
| Ae. albopictus | 7 | 8/8 (100) | 4/8 (50) | 9/10 (90) | 0/9 (0) |
| | 14 | 8/8 (100) | 7/8 (88) | 9/10 (90) | 1/9 (11) |

SG, salivary glands.

### Example 15

### Study to Determine Factor Affecting Vector Specificity and Epidemic Potential

### Viruses and Plasmids

The viruses and plasmids encoding full length infectious clones of the LR2006 OPY 1 strain CHIK-LR ic (GenBank accession number EU224268; http://www.ncbi.nlm.nih.gov/Genbank/index.html) and GFP-expressing full-length clone LR-GFP-226V (CHIK-LR 59GFP, GenBank accession number EU224269) have been previously described (Tsetsarkin et al., 2006; Vanlandingham et al., 2005). The plasmids 37997-226A (pCHIK-37997ic, GenBank accession number EU224270) encoding full-length infectious clones of the West African strain of CHIKV 37997 and a GFP-expressing full-length clone 37997-GFP-226A (pCHIK-37997-5GFP, GenBank accession number EU224271) were derived from previously described plasmids pCHIKic and 59CHIK EGFP (Vanlandingham et al., 2005) by introducing CHIKV encoding cDNA into a modified pSinRep5 (Invitrogen) at positions 8055-9930. Viruses derived from 37997-226A and 37997-GFP-226A are identical to viruses derived form pCHIKic and 59CHIK EGFP. To facilitate rapid screening of viruses in mosquitoes, the gene encoding enhance green fluorescent protein (eGFP), that is known not to compromise CHIKV phenotype in mosquitoes (Tsetsarkin et al., 2006), was incorporated into clones as previously described (Tsetsarkin et al., 2006). Plasmids were constructed and propagated using conventional PCR-based cloning methods (Sambrook et al., 1989). The entire PCR-generated regions of all constructs were verified by sequence analysis. For studies comparing the relative fitness of the mutant (E1-226V) virus and the pre-epidemic genotype (E1-226A), a silent mutation (6454C) was introduced into the CHIK-LR ic, to add an ApaI restriction site into the coding sequence of CHIK-LR ic. The resultant plasmid was designated LR-ApaI-226V. The E1-V226A mutation was introduced into CHIK-LR ic and LR-GFP-226V to generate plasmids designated as LR-226A and LR-GFP-226A, respectively. The mutation E1-A226V was also introduced into plasmids 37997-226A and 37997-GFP-226A. The resulted plasmids were designated 37997-226V and 37997-GFP-226V.

All plasmids were purified by centrifugation in CsCl gradients, linearized with NotI and in vitro transcribed from the minimal SP6 promoter using the mMESSAGE mMACHINE kit (Ambion) following the manufacturer's instructions. The yield and integrity of synthesized RNA were analyzed by agarose gel electrophoresis in the presence of 0.25 lg/ml of ethidium bromide. RNA (10 μg) was transfected into 1×10⁷ BHK-21 cells by electroporation as previously described (Tsetsarkin et al., 2006). Cells were transferred to 25 cm² tissue culture flasks with 10 ml of Leibovitz L-15 (L-15) medium, and supernatants were collected at 24 and 48 h post-electroporation and stored at 80° C. In parallel, 1×10⁵ electroporated BHK-21 cells were serially 10-fold diluted and seeded in six-well plates for infectious centers assay as previously described (Tsetsarkin et al., 2006).

### Cells and Mosquitoes:

BHK-21 (baby hamster kidney) cells were maintained at 37° C. in L-15 medium supplemented with 10% fetal bovine serum (FBS), 100 U penicillin, and 100 lg/ml streptomycin. C6/36 cells (Ae. albopictus) were grown in the same medium at 28 8C. Ae. aegypti (white-eyed Higgs variant of the Rexville D strain) and Ae. albopictus (Galveston strain) were reared at 27° C. and 80% relative humidity under a 16 h light: 8 h dark photoperiod, as previously described (Vanlandingham et al., 2005). Adults were kept in paper cartons supplied with 10% sucrose on cotton balls. To promote egg production females were fed on anaesthetized hamsters once per week. Rexville D strain of Ae. aegypti mosquitoes were originally selected for susceptibility to flavivirus infection (Miller and Mitchell, 1991). Since there are no known consequences of this original selection with respect to susceptibility to CHIKV, a white eyed variant of the strain that facilitates detection of GFP was used in these experiments.

### In Vitro Virus Growth of CHIKV in Standard and Cholesterol-Depleted C6/36 Cells:

To investigate if the mutation influenced cholesterol dependence of the virus, cholesterol-depleted C6/36 cells were prepared by five passages in L-15 medium containing 10% FBS treated with 2% CAB-O-Sil (Acros Organics) for 12 h at room temperature as described (Weinstein, 1979). CHIKV growth curves were determined by infecting cholesterol-depleted and normal C6/36 cells at a multiplicity of infection (MOI) of 0.1 and 1.0, respectively, by rocking for 1 h at 25° C. The cells were washed three times with L-15 medium and 5.5 ml of fresh L-15 supplied with 10% of standard or CAB-O-Sil treated FBS was added to the flask. At the indicated times post-infection, 0.5 ml of medium was removed and stored at 80° C. until titrated. The volume of medium was then restored by adding 0.5 ml of appropriate medium.

### Titrations:

Viral titers from mosquito samples and from tissue culture supernatant were determined using Vero cells and expressed as tissue culture infectious dose 50 percent endpoint titers (Log₁₀TCID₅₀) as previously described (Higgs et al., 1997). Additionally, for viral competition experiments, titers of LR-Apa-226V LR-226A viruses were determined using standard plaque assay on Vero cells as previously described (Lemm et al., 1990).

### Oral Infection of Mosquitoes:

Ae. aegypti and Ae. albopictus were infected in an Arthropod Containment Level 3 insectary as described previously (Vanlandingham et al., 2005; McElroy et al., 2006). To make infectious blood meals for the viruses lacking eGFP, viral stocks derived from electroporated BHK-21 cells were mixed with an equal volume of defibrinated sheep blood and supplemented with 3 mM ATP as a phago-stimulant. To produce infectious blood meals for the eGFP-expressing viruses, the viruses were additionally passed on BHK-21 cells. The cells were infected at a MOI˜1.0 with virus derived from electroporation. At 2 dpi, cell culture supernatants were mixed with an equal volume of defibrinated sheep blood and presented to 4- to 5-day-old female mosquitoes that had been starved for 24 h, using a Hemotek membrane feeding system (Discovery Workshops) and hamster skin membrane. Mosquitoes were allowed to feed for 45 min, and engorged mosquitoes (stage≧3+(Pilitt and Jones, 1972)) were sorted and returned to a cage for maintenance. Blood meals and three to four mosquitoes were immediately removed for titration and/or RNA extraction. Depending on the purpose of the experiments, mosquitoes were collected at different days post-infection and either titrated to determine viral titer, dissected for analysis of eGFP expression in the midguts or salivary glands (Tsetsarkin et al., 2006), or used for RNA extraction in competition experiments.

To estimate the Oral Infectious Dose 50% values (OID₅₀), serial 10-fold dilutions of viruses were made in L-15 medium followed by mixing the samples with defibrinated sheep blood. Mosquitoes were dissected at 7 dpi and eGFP expression in infected midguts was analyzed by fluorescence microscopy. A mosquito was considered infected if at least one foci of eGFP-expressing cells was present in the midgut. The experiments were performed twice for each virus. OID₅₀ values and confidence intervals were calculated using PriProbit (version 1.63).

### Viral Competition Experiments:

To test the hypothesis that the E1 A226V mutation might be associated with a competitive advantage in mosquito vectors, competition assays were designed similar to those described previously in mice (Pfeiffer and Kirkegaard, 2005), with minor modifications (FIG. 15A). Both Ae. aegypti and Ae. albopictus mosquitoes were presented with a blood meal containing 10⁷ plaque-forming units (pfu)/ml of LR-Apa-226V and 107 pfu/ml of LR-226A viruses. It had been previously found that for these two viruses the ratio of viral RNAs corresponds to the ratio of viral titers (data not shown). Midguts were collected at 7 dpi and analyzed in pools of eight to ten, and heads were collected at 12 dpi and analyzed in pools of five.

RNA was extracted from the tissue pools using TRIzol reagent (Invitrogen) followed by additional purification using a Viral RNA mini kit (QIAGEN). RNAs from blood meal samples were extracted using Viral RNA Mini Kit followed by treatment with DNAse (Ambion) to destroy any residual plasmid DNA contaminant in the viral samples. RNA was reversed transcribed from random hexamer primers using Superscript III (Invitrogen) according to the manufacturer's instructions. cDNA was amplified from 41855 ns-F5 (59-ATATCTAGACATGGTGGAC; SEQ ID NO: 14) and 41855 ns-R1 (59-TATCAAAGGAGGCTATGTC; SEQ ID NO: 15) primers using Taq DNA polymerase (New England Biolabs). PCR products were purified using Zymo clean columns (Zymo Research) and were quantified by spectrophotometry. Equal amount of PCR products were digested with ApaI, separated in 2% agarose gels that were stained using ethidium bromide. Thus the LR-Apa-226V and LR-226A viruses could be distinguished by size on an agarose gel (FIG. 15A). Gel images were analyzed using TolaLab (version 2.01). Relative fitness of LR-Apa-226V and LR-226A viruses was calculated as a ratio between 226V and 226A bands in the sample divided by the control ratio of 226V and 226A in the blood meal.

### Virus Competition in an Animal Transmission Model:

Ae. aegypti and Ae. albopictus mosquitoes were presented with a blood meal containing 10⁷ pfu/ml of LR-Apa-226V and 10⁷ pfu/ml of LR-226A viruses. At 13 dpi, ten to 15 mosquitoes were placed in separate paper cartons and starved for 24 h. The next day the mosquitoes in each carton were presented with individual 2- to 3-day-old suckling mouse (Swiss Webster). Feeding continued until 2-3 mosquitoes per carton were fully engorged (stage≧3+(Pilitt and Jones, 1972). In a parallel experiment six 2- to 3-day-old suckling mice were subcutaneously infected with 20 μl of mixture containing ˜25 pfu of LR-Apa-226V and ˜25 pfu of LR-226A viruses. Mice were returned to their cage and sacrificed on day 3 post-exposure. Blood from each individual mouse (˜50 μl) was collected and immediately mixed with 450 μl of TRIzol reagent for RNA extraction. The RNA was processed as described above.

Effect of E1 A226V Mutation on Fitness of CHIKV in Ae. albopictus Mosquitoes:

To test the hypothesis that the E1-A226V mutation altered CHIKV infectivity for Ae. albopictus mosquitoes, CHIKV infectious clones derived from an epidemic Reunion island human isolate were used (Vanlandingham et al., 2005), including one clone (LR-GFP-226V) expressing enhanced green fluorescent protein (eGFP). Clones were further engineered to express E1 protein containing an alanine at position E1-226 (LR-GFP-226A) representing the CHIKV genotype prevalent prior to the outbreak gaining momentum. RNAs produced from both clones (LR-GFP-226V and LR-GFP-226A) have comparable specific infectivity values, produced similar viral titers following transfection into BHK-21 cells and had similar growth kinetics in mosquito (C6/36) and mammalian (BHK-21) cells lines.

The relative infectivity of LR-GFP-226V and LR-GFP-226A viruses was analyzed in female Ae. albopictus mosquitoes orally exposed to serial 10-fold dilutions of CHIKV (LR-GFP-226 V or A). To determine whether infection rates correlate with blood meal titer, midguts dissected from mosquitoes at 7 days post-infection (dpi) were analyzed for foci of eGFP-expressing cells by fluorescence microscopy (FIG. 14A; Table 9). In two independent experiments, LR-GFP-226V virus was found to be approximately 100-fold more infectious to Ae. albopictus than LR-GFP-226A virus (p,0.01). To test if the infectivity phenotype was directly linked to the mutation, the complementary reverse mutation, E1-A226V, was introduced into an infectious clone of a West African CHIKV strain, 37997-GFP (37997-GFP-226A). The Reunion and 37997 strains of CHIKV are distantly related, with only 85% nucleotide sequence identity. The parental 37997-GFP-226A and the 37997-GFP-226V viruses were indistinguishable in cell culture experiments; however, in vivo experiments in Ae. albopictus mosquitoes revealed that the E1-A226V mutation significantly decreases the oral infectious dose 50 (OID50) value for the 37997-GFP-226V virus (p<0.01) to an extent similar to that observed for LR-GFP-226V virus (FIG. 14B; Table 9). These data conclusively demonstrate that the single E1-A226V point mutation is therefore sufficient to significantly reduce the OID₅₀ of the 37997-GFP virus (p,0.01) in Ae. albopictus mosquitoes equivalent to that observed for the LR-GFP-226V virus (FIG. 14A; Table 9).

TABLE 9
Log₁₀OID₅₀/ml for CHIKV in Ae. albopictus mosquitoes.

| | | | Mosquitoes | | |
|---|---|---|---|---|---|
| Backbone | Exp^{a} | Virus | analyzed | Log₁₀ OID₅₀ Cl₉₅^{c} | p value |
| CHIK Reunion | 1 | LR-GFP-226V | 98 | <4.22 | p < 0.01 |
| | | LR-GFP-226A | 101 | 5.42 ± 0.29 | |
| | 2 | LR-GFP-226V | 171 | 3.52 ± 0.28 | p < 0.01 |
| | | LR-GFP-226A | 93 | 5.48 ± 0.23 | |
| CHIK 37997 | 1 | 37997-GFP-226V | 131 | 5.20 ± 0.22 | p < 0.01 |
| | | 37997-GFP-226A | 138 | 3.31 ± 0.42 | |
| | 2 | 37997-GFP-226V | 129 | 4.90 ± 0.25 | p < 0.01 |
| | | 37997-GFP-226A | 136 | 3.06 ± 0.32 | |

OID₅₀ values and confidence intervals were calculated using PriProbit (version 1.63).
^{a}Experiment number.
^{b}Number of mosquitoes used to estimate Log₁₀OID₅₀/ml.
^{c}95% confidence intervals.

To further evaluate viral fitness of the epidemic CHIKV E1-A226V mutation in Ae. albopictus, viral competition experiments were performed. Although the CHIKV eGFP-expressing infectious clones of the present invention have similar infection properties in mosquitoes as wild-type viruses (Tsetsarkin et al., 2006; Vanlandingham et al., 2005), to address potential concerns that eGFP expression might influence OID₅₀ values, LR-226A and LR-ApaI-226V viruses were constructed without eGFP and used in viral competition experiments (FIG. 15A). LR-ApaI-226V was derived from previously described CHIK-LR ic, by the introduction of a silent marker mutation, A6454C, in order to add an ApaI restriction site into the coding sequence. It was shown that the A6454C mutation does not affect the specific infectivity value, the viral titer after RNA transfection into BHK-21 cells value, the viral growth kinetics in BHK-21 and C6/36 cells, infectivity for and viral titers in Ae. aegypti and Ae. albopictus mosquitoes or viral fitness for growth in BHK-21 and C6/36 cells as determined by competition assay. These data indicate that the introduced mutation is indeed silent and does not affect the fitness of LR-ApaI-226V.

For viral competition experiments, LR-ApaI-226V virus (10⁷pfu) was mixed with an equal amount of LR-226A virus. LR-ApaI-226V and LR-226A viruses are indistinguishable in cell culture experiments. Mixtures of LR-ApaI-226V and LR-226A viruses were orally presented to Ae. albopictus mosquitoes in a blood meal, and midguts were examined at 7 dpi. The relative amount of RNA derived from LR-ApaI-226V in the midgut cells increased 5.760.6 times as compared to the initial relative amount of LR-ApaI-226V RNA in the blood meal sample (FIG. 15B). These data support the observation that the E1-A226V mutation enhances infectivity of CHIKV for Ae. albopictus mosquitoes and furthermore demonstrate that the mutation could provide an evolutionary advantage over E1-226A viruses in an atypical vector and may have perpetuated the outbreak in a region where Ae. albopictus was the predominant anthropophilic mosquito species.

To determine if the enhanced midgut infectivity associated with the E1-A226V mutation may result in more efficient viral dissemination into secondary tissues, the kinetics of viral dissemination by LR-GFP-226V and LR-GFP-226A into salivary glands, and competition between LR-ApaI-226V and LR-226A for dissemination into mosquito heads were analyzed (FIGS. 16A and 16B). LR-GFP-226V virus disseminated more rapidly into Ae. albopictus salivary glands at all time points, with a significant difference at 7 dpi (p1/40.044, Fisher's exact test). Similarly, in three of four replicates of competition experiments, RNA from LR-ApaI-226V virus was dramatically more abundant in the heads of Ae. albopictus mosquitoes as compared to RNA from LR-226A (FIG. 16B, lines 1, 3, 4), although in one replica LR-ApaI-226V RNA was only slightly more abundant as compared to the initial viral RNA ratio (FIG. 16B, line 2). This variability of the results may be due to random pooling of mosquito heads. Thus, replicate two may have included more heads negative for LR Apal-226V relative to heads positive for LR-226A RNA.

Another possibility is that at some point during viral dissemination from the midguts into mosquito heads, LR-226A may replicate more rapidly than LR-ApaI-226V. To further investigate this relationship, Ae. albopictus mosquitoes were orally presented with either LR-ApaI-226V or LR-226A and whole mosquito body viral titers were compared at different time points pi. Surprisingly, no significant differences between viral titers were found, with the exception of 1 dpi, where the LR-ApaI-226V titer was 0.5 Log₁₀ tissues culture infectious dose 50 percent end point titer (Log₁₀, TCID₅₀/mosquito) higher than of the LR-226A titer (FIG. 17A). This may be due to more efficient colonization of Ae. albopictus midguts by LR-ApaI-226V. The absence of significant differences in viral titers at later time points may be due to variation in viral titers among individual mosquitoes. Competition between LR-ApaI-226V and LR-226A was analyzed at different time points in order to investigate the relationship between replication of LR-ApaI-226V and LR-226A viruses in Ae. albopictus mosquitoes (FIG. 17B).

As expected, the viral RNA from LR-ApaI-226V was predominant at the early time points of 1 and 3 dpi. Interestingly, between 3 and 5 dpi the viral RNA ratio shifted toward LR-226A virus indicating that at these time points, LR-226A replicates more efficiently in some mosquito tissues (FIG. 17B). This short period of time may have a slight effect on the overall outcome of competition for dissemination into salivary glands because there is a reverse shift in the RNA ratio between days 5 and 7 toward LR-ApaI-226V virus, which continues through 14 dpi. These data indicate that the E1-A226V mutation not only increases midgut infectivity but also is associated with more efficient viral dissemination from the midgut into secondary organs, suggesting that the E1-A226V mutation would increase transmissibility of CHIKV by Ae. albopictus mosquitoes.

A competition assay between LR-ApaI-226V and LR-226A viruses was used to examine transmission by Ae. albopictus to suckling mice to assess the potential for the E1-A226V mutation to influence virus transmission. Ae. albopictus mosquitoes were orally presented with a mixture of LRApaI-226V and LR-226A viruses and at 14 dpi were allowed to feed on suckling mice. Mice were sacrificed and bled on day 3 following exposure and the presence of CHIKV RNA in the blood was analyzed by RT-PCR followed by restriction digestion with ApaI (FIG. 18B). Blood obtained from 100% of experimental mice contained detectable amounts of viral RNA, indicating that virus was transmitted by Ae. albopictus mosquitoes to suckling mice. More importantly, in all six mice analyzed, RNA derived from LR-ApaI-226V was the predominant viral RNA species, indicating that under the conditions of competition for transmission, the E1-A226V mutation directly increases CHIKV transmission by Ae. albopictus mosquitoes. Interestingly, in the control experiment in which mice were subcutaneously inoculated with ˜50 pfu of 1:1 mixture of LR-ApaI-226V and LR-226A viruses, RNAs from both viruses were readily detected and no difference was observed in the viral RNA ratio 3 dpi (FIG. 18A) indicating that at least in mice, E1-A226V is not associated with changes

Effect of E1 A226V Mutation on Fitness of CHIKV in Ae. aegypti Mosquitoes:

Since the E1-A226V mutation confers a fitness advantage in Ae. albopictus, it is unknown why this mutation had not been observed previously. It is possible that this change might have a deleterious effect on viral fitness in the vertebrate host, although the data of direct competition of LR-ApaI-226V and LR-226A viruses in suckling mice (FIG. 18A) and analysis of CHIKV cellular tropism of four clinical isolates from Reunion (which have either A or V at position E1-226) (Sourisseau et al., 2007) suggest that this is unlikely. An alternative hypothesis is that the E1-A226V mutation might compromise the fitness of CHIKV or have neutral fitness effects in the mosquito species which served as a vector for CHIKV prior to its emergence on Reunion island. Since Ae. aegypti has generally been regarded as the main vector for CHIKV prior to the emergence on Reunion island, the effect of the E1-A226V mutation on fitness of CHIKV in Ae. aegypti was analyzed herein.

In contrast to the results obtained in Ae. albopictus mosquitoes, OID₅₀ values of viruses containing the E1-226V in the backbone of the Reunion and 37997 strains of CHIKV were approximately 0.5 Log₁₀OID₅₀/ml higher than the OID₅₀ values of E1-226A viruses in all experiments using Ae. aegypti. These differences were statistically significant for one out of two replicates for each virus pair (FIGS. 14C and 14D; Table 10). A competition assay examining LR-ApaI-226V and LR-226A virus infection in Ae. aegypti midguts, demonstrated that LR-226A virus out-competed LR-ApaI-226V virus at 7 dpi in all four replicates using ten midguts per replicate and that the amount of LR-226A RNA increased on average 3.1 times as compared to the initial blood meal RNA ratio (FIG. 15C). These data suggest that the E1-A226V mutation has a slight negative effect on CHIKV infectivity of Ae. aegypti midguts.

TABLE 10
Log₁₀OID₅₀/ml for CHIKV in Ae. albopictus mosquitoes.

| | | | Mosquitoes | | |
|---|---|---|---|---|---|
| Backbone | Exp^{a} | Virus | analyzed | Log₁₀ OID₅₀ Cl₉₅^{c} | p value |
| CHIK Reunion | 1 | LR-GFP-226V | 65 | 6.77 ± 0.40 | p < 0.1 |
| | | LR-GFP-226A | 103 | 6.12 ± 0.28 | |
| | 2 | LR-GFP-226V | 107 | 6.26 ± 0.30 | p < 0.05 |
| | | LR-GFP-226A | 53 | 5.62 ± 0.33 | |
| CHIK 37997 | 1 | 37997-GFP-226V | 161 | 5.77 ± 0.25 | p < 0.01 |
| | | 37997-GFP-226A | 162 | 6.59 ± 0.34 | |
| | 2 | 37997-GFP-226V | 136 | 5.83 ± 0.30 | p < 0.1 |
| | | 37997-GFP-226A | 127 | 6.34 ± 0.29 | |

OID₅₀ values and confidence intervals were calculated using PriProbit (version 1.63).
^{a}Experiment number.
^{b}Number of mosquitoes used to estimate Log₁₀OID₅₀/ml.
^{c}95% confidence intervals.

The effect of the E1-A226V mutation on the ability of CHIKV to disseminate into Ae. aegypti secondary organs was also analyzed (FIGS. 16C and 16D). LR-GFP-226V and LR-GFP 226A viruses both have similar kinetics of dissemination into salivary glands following oral infection using titers 1-2 Log₁₀TCID₅₀ higher than their OID₅₀ value in Ae. aegypti (FIG. 16C). In a competition assay, both LR-ApaI-226V and LR-226A viruses disseminated similarly into the heads of Ae. aegypti. In two of four replicas, there was a slight increase in the relative amount of LR-226A RNA (FIG. 16D, lines 1, 4); whereas the other two replicas showed a decrease in LR-226A RNA (FIG. 15D, lines 2, 3), relative to the initial ratio of the RNA of LR-ApaI-226V and LR-226A viruses in the blood meal. A competition of LR-ApaI-226V and LR-226A viruses for transmission by Ae. aegypti to suckling mice was also analyzed (FIG. 18C). In contrast to transmission by Ae. albopictus mosquitoes, five out of six mice fed upon by Ae. aegypti contained comparable amounts of RNA derived from both viruses and only one out of six mice contained RNA derived exclusively from LR-ApaI-226V.

### E1-A226V Mutation Modulates Cholesterol Dependence of CHIKV

It has been previously shown that a P→S mutation in the same E1-226 position of SFV releases cholesterol dependence of the virus in C6/36 cells (Vashishtha et al., 1998) and results in significantly more rapid growth of SFV in Ae. albopictus mosquitoes after intrathoracic inoculation (Ahn et al., 1999). To determine if a requirement for cholesterol in the cell membrane is important for CHIKV, cholesterol dependence of CHIKV E1-226A and E1-226V viruses was analyzed herein (FIGS. 19A-19B). Growth curves of E1-226A and E1-226V viruses in the background of Indian Ocean and West African strains of CHIKV were almost indistinguishable when grown in C6/36 cells maintained in L-15 supplied with standard 10% FBS (FIG. 19A). However, when the cells were depleted of cholesterol, LR-226A and 37997-226A viruses replicated significantly more rapidly than LR-226V and 37997-226V viruses, reaching 3 Log₁₀TCID₅₀/ml higher titer at 1, 2 and 3 dpi (FIG. 19B). These data indicate that adaptation of CHIKV to Ae. albopictus mosquitoes coincides with CHIKV dependence on cholesterol in the target cell membrane.

The following references were cited herein:
Adelman, Z. N. et al., 2001, Insect Mol. Biol. 10:265-273.
Ahn et al., 1999, Virology, 262: 452-456.
Aranda C et al., 2006, Med Vet Entomol. 20: 150-152.
Arankalle et al., 2007, J Gen Virol 88: 1967-1976.
Attardo, G. M. et al., 2003, Proc. Natl. Acad. Sci. U.S.A 100:13374 13379.
Binn et al., 1967, Am J Trop Med Hyg 16: 782-785.
Bowen, M. D. 1987. Transovarial transmission of arboviruses by their vectors: experimental transovarial transmission of sindbis virus (genus Alphavirus) by Aedes aegypti. Thesis. London School of Hygiene and Tropical Medicine.
Brault, A. C., et al., 2004, Insect Mol. Biol. 13:625-635.
Carletti et al., 2007, Am J Trop Med Hyg 77: 521-524.
Carey et al., 1969 Trabs R Soc Trop Med Hyg 63: 434-445.
Cary, L. C., et al., 1989, Virology 172:156-169.
Chatterjee et al., 2000, J Virol 74: 1623-1631.
Cheng, L. L., et al., 2001, J. Insect Sci. 1:10.
Coates, C. J., et al., 1998. Proc. Natl. Acad. Sci. U.S.A 95:3748-3751.
Cosgrove, J. B., et al., 1994, J. Am. Mosq. Control Assoc. 10:434-436.
Foy, B. D., et al., 2004, Insect Mol. Biol. 13:89-100.
Franz, G. and C. Savakis, 1991, Nucleic Acids Res. 19:6646.
Frohman, M. A. 1994, PCR Methods Appl. 4:S40-S58.
Gould, E. A., et al., 1985a, J. Virol. Methods 11:41-48.
Gould, E. A., et al., 1985b, J. Gen. Virol. 66 (Pt 7):1369-1382.
Griffin D E. 2001. Sindbis virus. In: Service MW, editor. The Encyclopedia of Arthropod-transmitted Infections. Oxford: CABI Publishing. p 469-473.
Grossman, G. L., et al., 2001, Insect Mol. Biol. 10:597-604.
Hahn, C. S., et al., 1992, Proc. Natl. Acad. Sci. U.S.A 89:2679-2683.
Hart, M. K. et al., 2001, Vaccine 20:616-622.
Higgs S. 2006. Vector-Borne Zoonotic Dis 5:1.
Higgs, S., et al., 1997. Viral expression systems and viral infections in insects. Pages 457-483 in Crampton, Beard, and Louis, eds. The Molecular Biology of Disease Vectors: A Methods Manual. Chapman and Hall, UK.
Higgs, S., et al., 1995. Insect Mol. Biol. 4:97-103.
Higgs, S., et al., 1999. Biotechniques 27:908-911.
Higgs, S., et al., 1998. Am. J. Trop. Med. Hyg. 58:663-670.
Hsieh et al., 1984. J Biol Chem 259: 2375-2382.
Jacobson, J. W. and D. L. Hartl. 1985, Genetics 111:57-65.
James, A. A., et al., 1999, Parassitologia 41:461-471.
Jasinskiene, N., et al., 1998, Proc. Natl. Acad. Sci. U.S.A 95:3743-3747.
Jasinskiene, N., et al., 2000, Insect Mol. Biol. 9:11-18.
Johnson, B. W., et al., 1999. Proc. Natl. Acad. Sci. U.S.A 96:13399-13403.
Karabatsos, N. International Catalogue of Arboviruses. Third Ed. 1985. San Antonio, Tex., Am. Society of Tropical Medicine and Hygiene. Ref Type: Catalog Miller, B. R. and C. J. Mitchell, 1991, Am. J. Trop. Med. Hyg. 45:399-407.
Kielan and rey, 2006, Nat Rev Microbiol 4: 67-76.
Klimstra et al., 2003. J Virol. 77: 12022-12032.
Kolokoltsov et al., 2006, Virology 347: 333-342.
Kuhn et al., 1990. J Virol 64: 1465-1476.
Lemm et al., 1990, J Virol 64: 3001-3011.
Liljestrom et al., 1991, J Virol 65: 4107-4113.
Lu et al., 1999, J Virol 73: 4272-4278.
McElroy et al., 2006, J Gen Virol 87: 2993-3001.
Miller and Mitchell, 1991, Am J Trop Med Hyg 45: 399-407.
Olson, K. E., et al., 1996, Science 272:884-886.
Olson, K. E., et al., 1994, Insect Biochem. Mol. Biol. 24:39-48.
Olson, K. E., et al., 2000, Insect Mol. Biol. 9:57-65.
Parola et al., 2006, Emerg Infect Dis. 12: 1393-1399.
Paul and Singh, 1968, Indian J Med Res 56: 802-811.
Perera, O. P., et al., 2002, Insect Mol. Biol. 11:291-297.
Pfeiffer and Kirkegaard, 2005, PLos Pathog 1: e11.
Pierro, D. J., et al., 2003, Insect Mol. Biol. 12:107-116.
Pilitt and Jones, 1972, J Med Entomol 9: 334-337.
Pinkerton, A. C., et al., 2000, Insect Mol. Biol. 9:1-10. Powers, A. M., et al., 1996, Proc. Natl. Acad. Sci. U.S.A 93:4187-4191.
Powers et al., 2000, J Gen Virol, 81: 471-479.
Pudney, M., et al., 1979. Replication of arboviruses in arthropod in vitro systems. In: Edouard Kurstak (Ed.), Arctic and Tropical Arboviruses. Academic Press, New York, pp. 245-262.
Pugachev et al., 1995. Virology 212: 587-594.
Raymes-Keller, A. et al., 1995, Insect Molecular Biology. 4:245-251.
Rice et al., 1987, J Virol, 61: 3809-3819.
Sambrook et al., 1989, Molecular Cloning: a Laboratory manual. Cold Spring Harbor (New York): Cold Spring Harbor Laboratory.
Schuffenecker et al., 2006, PLoS Med 3: e263.
Schlesinger S, Schlesinger M J. 2001. Togaviridae: The Viruses and Their Replication. In: Knipe D M, Howley P M, editors. Fundamental Virology. Philadelphia: Lippincott Williams and Wilkins. p 567-588.
Seabaugh, R. C., et al., 1998, Virology 243:99-112.
Shiao, S. H., et al., 2001, Insect Mol. Biol. 10:315-321.
Sourisseau et al., 2007, PLos Pathog. 3: e89.
Strauss, J. H. and E. G. Strauss, 1994, Microbiol. Rev. 58:491-562.
Tamang, D., et al., 2004, Insect Mol. Biol. 13:595-602.
Tesh et al., 1976, Am J Trop Med Hyg 25: 326-335.
Tsetsarkin et al, 2006, Vector Borne Zoonotic Dis 6: 325-337.
Turell et al., 1992. J Med Entomol. 29: 49-53.
Vanlandingham et al., 2005a, Insect Biochem Mol Biol. 35: 1162-1170.
Vanlandingham, D. L., et al., 2005 or 2005b, Am. J. Trop. Med. Hyg. 72:616-621.
Vanlandingham, D. L., et al., 2006, Am. J. Trop. Med. Hyg. 74:663-669.
Vashishtha et al., 1998, J Cell Biol. 140: 91-99.
Warren, W. D., et al., 1994, Genet. Res. 64:87-97.
Wendell, M. D., et al., 2000, Insect Mol. Biol. 9:119-125.
Weinstein, 1979, Circulation 60: 204.
Wolfe et al., 2007, Nature 447: 279-283.
Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

What is claimed is:

1. An expression vector, comprising: a DNA sequence encoding a full-length chikungunya virus comprising non-structural protein genes and structural protein genes of the chikungunya virus.

2. The expression vector of claim 1, wherein the DNA sequence encoding the non-structural protein genes is inserted in one plasmid and the DNA sequence encoding the structural protein genes is inserted in a second plasmid.

3. The expression vector of claim 1, wherein the DNA sequence encoding the non-structural protein genes is inserted in one plasmid, the DNA sequence encoding the capsid structural protein genes is inserted in a second plasmid and the DNA sequence encoding the rest of the structural protein genes is inserted in a third plasmid.

4. The expression vector of claim 1, wherein the structural protein gene(s) comprises a single amino acid substitution effective to increase infectivity of an chikungunya virus infectious clone in a mosquito.

5. The expression vector of claim 4, wherein the amino acid substitution comprises A226V mutation in E1 protein of the chikungunya virus.

6. The expression vector of claim 1, wherein the chikungunya virus DNA sequence is derived from 37997 strain, Nagpur (India) 653496 strain, S27-African prototype strain, Ross strain or LR2006 isolates from LaReunion strain of chikungunya virus.

7. The expression vector of claim 1, further comprising: a heterologous gene, a knock-out gene, an over-expressing gene or an immunogenic sequence.

8. A host cell comprising and expressing the expression vector of claim 1.

9. An infectious clone comprising the DNA of claim 1.

10. The infectious clone of claim 9, wherein the clone encodes an attenuated chikungunya virus.

11. A pharmaceutical composition comprising:
the attenuated chikungunya virus encoded by the clone of claim 9, wherein said clone does not comprise an amino acid substitution in the structural protein gene(s), a DNA sequence encoding a protein of interest expressed by the clone and a pharmaceutically acceptable carrier.

12. The pharmaceutical composition of claim 11, wherein the DNA sequence encoding the protein of interest is the DNA sequence of a heterologous gene, an over-expressed gene, knockout/knockdown gene or an immunogenic peptide.

13. An immunogenic composition comprising:
a live attenuated chikungunya virus encoded by a clone of claim 9, wherein said clone does not comprise an amino acid substitution in the structural protein gene(s), and a DNA sequence encoding an immunogenic peptide expressed by the clone.

14. An immunogenic composition, comprising:
an attenuated chikungunya virus encoded by clone of claim 9, wherein said clone does not comprise an amino acid substitution in the structural protein gene(s), wherein the attenuated chikungunya virus is inactivated and a DNA sequence encoding an immunogenic peptide expressed by the clone.

15. A method of evaluating function of a gene in an organism, comprising:
expressing the gene or knocking out the gene of interest using the clone of claim 9; and
determining the effect of over-expressing or knocking out the gene in the organism, thereby evaluating the function of the gene in the organism.

16. A method of inducing a protective immune response in a subject, comprising:
administering pharmaceutically effective amounts of the immunogenic composition of claim 13, thereby inducing a protective immune response in the subject.

17. The method of claim 16, wherein the subject is a human or a non-human primate.

18. A method of inducing protective immune response in a subject, comprising:
administering pharmaceutically effective amounts of an immunogenic composition of claim 14, thereby inducing protective immune response in the subject.

19. The method of claim 18, wherein the subject is a human or a non-human primate.

20. An expression vector, comprising:
a DNA sequences encoding a full-length chikungunya virus comprising non-structural protein genes and structural protein genes of the chikungunya virus and an additional subgenomic promoter.

21. The expression vector of claim 20, wherein the DNA sequence encoding the non-structural protein genes is inserted in one plasmid and the DNA sequence encoding the structural protein genes and the subgenomic promoter is inserted in a second plasmid.

22. The expression vector of claim 20, wherein the DNA sequence encoding the non-structural protein genes is inserted in one plasmid, the DNA sequence encoding the capsid structural protein genes is inserted in a second plasmid and the DNA sequence encoding the rest of the structural protein genes and the sub-genomic promoter is inserted in a third plasmid.

23. The expression vector of claim 20, wherein the additional subgenomic promoter is placed either 3′ or 5′ to the structural protein genes.

24. The expression vector of claim 20, wherein the structural protein gene(s) comprises a single amino acid substitution effective to increase infectivity of an chikungunya virus infectious clone in a mosquito.

25. The expression vector of claim 24, wherein the amino acid substitution comprises A226V mutation in E1 protein of the chikungunya virus.

26. The expression vector of claim 20, wherein the chikungunya virus DNA sequence is derived from 37997 strain, Nagpur (India) 653496 strain, S27-African prototype strain, Ross strain, LR2006 isolates from LaReunion strain of chikungunya virus.

27. The expression vector of claim 20, further comprising:
a heterologous gene, a knock-out gene, an over-expressing gene or an immunogenic sequence.

28. A host cell comprising and expressing the vector of claim 20.

29. An infectious clone comprising the DNA of claim 20.

30. The infectious clone of claim 29, wherein the clone encodes an attenuated chikungunya virus.

31. A pharmaceutical composition comprising:
an attenuated chikungunya virus and a subgenomic promoter encoded by the clone of claim 29, wherein said clone does not comprise an amino acid substitution in the structural protein gene(s), DNA sequence encoding a protein of interest expressed by the clone and a pharmaceutically acceptable carrier.

32. The pharmaceutical composition of claim 31, wherein the DNA sequence encoding the protein of interest is the DNA sequence of a heterologous gene, an over-expressed gene, knockout/knockdown gene or an immunogenic peptide.

33. An immunogenic composition, comprising:
a live attenuated chikungunya virus and a sub-genomic promoter encoded by clone of claim 29, wherein said clone does not comprise an amino acid substitution in the structural protein gene(s), and a DNA sequence encoding an immunogenic peptide expressed by the clone.

34. An immunogenic composition, comprising:
an attenuated chikungunya virus and a sub-genomic promoter encoded by clone of claim 29, wherein said clone does not comprise an amino acid substitution in the structural protein gene(s), wherein the attenuated chikungunya virus is inactivated and a DNA sequence encoding an immunogenic peptide expressed by the clone.

35. A method of evaluating function of a gene in an organism, comprising:
expressing the gene or knocking out the gene of interest using the clone of claim 29; and
determining the effect of over-expressing or knocking out the gene in the organism, thereby evaluating the function of the gene in the organism.

36. A method of inducing protective immune response in a subject, comprising:
administering pharmaceutically effective amounts of the immunogenic composition of claim 33, thereby inducing a protective immune response in the subject.

37. The method of claim 36, wherein the subject is a human or a non-human primate.

38. A method of inducing a protective immune response in a subject, comprising the step of administering pharmaceutically effective amounts of the immunogenic composition of claim 34, thereby inducing protective immune response in the subject.

39. The method of claim 38, wherein the subject is a human or a non-human primate.

40. A chikungunya virus replicon system, comprising:
a replicon comprising non-structural genes of the chikungunya virus and a marker gene; and
a helper system comprising structural genes of the chikungunya virus.

41. The chikungunya virus replicon system of claim 40, wherein the marker gene is a gene encoding green fluorescent protein.

42. The chikungunya virus replicon system of claim 40, wherein the structural protein gene(s) comprises a single amino acid substitution effective to increase infectivity of an chikungunya virus infectious clone in a mosquito.

43. The chikungunya virus replicon system of claim 42, wherein the amino acid substitution comprises A226V mutation in E1 protein of the chikungunya virus.

44. The chikungunya virus replicon system of claim 40, wherein the non-structural and structural genes of the chikungunya virus are derived from chikungunya virus 37997 strain, chikungunya virus Nagpur (India) 653496 strain, S27-African prototype strain of chikungunya virus, Ross strain of chikungunya virus or LR2006 isolates from LaReunion strain of chikungunya virus.

45. A host cell, comprising and expressing the chikungunya virus replicon system of claim 40.

46. A virus like particle, comprising:
genes encoded by the replicon system of claim 40.

47. A method of identifying sites of primary chikungunya virus infection in a mosquito vector, comprising:
feeding the virus like particle of claim 46 to the mosquito vector; and
detecting expression of the marker gene in the midgut and salivary gland of the mosquito vector, thereby identifying sites of primary chikungunya virus infection in the mosquito vector.
